(19) United States
(12) Patent Application Publication
(10) Pub. No.: US 2015/0030583 A1
(43) Pub. Date: Jan. 29, 2015

(54) Methods of Treating Serosal Cancer

(71) Applicants: Malcolm A.S. Moore, New York, NY (US); Server A. Ertem, New York, NY (US)

(72) Inventors: Malcolm A.S. Moore, New York, NY (US); Server A. Ertem, New York, NY (US)

(73) Assignee: SLOAN KETTERING INSTITUTE FOR CANCER RESEARCH, New York, NY (US)

(21) Appl. No.: 14/009,176

(22) PCT Filed: Mar. 30, 2012
(86) PCT No.: PCT/US12/31390

§ 371 Date: Aug. 27, 2014

Related U.S. Application Data
(60) Provisional application No. 61/470,958, filed on Apr. 1, 2011.

(51) Int. Cl. A61K 38/48; A61K 47/48; A61K 31/337; A61K 31/4045; A61N 5/10; A61K 31/165; A61K 31/365; A61K 31/03; G01N 33/50; A61K 38/47; A61K 31/5377

(52) U.S. Cl. CPC A61K 38/4886; A61K 38/47; A61K 47/48215; A61K 31/337; A61K 31/4045; A61K 31/5377; A61K 31/165; A61K 31/365; A61K 31/03; G01N 33/5011; A61N 5/1001; C12Y 302/01035; C12Y 304/24007; G01N 2800/52; G01N 2800/7028
USPC 424/94.62; 424/94.67; 514/419; 514/236.8; 514/629; 514/450; 514/753; 435/29; 435/18; 600/1

(57) ABSTRACT

The discovery of clonally pure populations of serosal cancer stem cells (CSCs) as well as methods of producing CSCs, culturing the CSCs and using them in screening assays, has lead to the development of methods of treating serosal and ovarian cancers by targeting removal or inhibition of the glycocalyx coat surrounding such cells, and includes combination therapies using particular chemotherapeutics in conjunction with glycocalyx inhibitors, as well as the same new chemotherapy treatments without targeting the glycocalyx, where the chemotherapeutic agent is any one of LBH-589 (Panobinostat), NVP-AUY922, LAQ824 (NVP-LAQ824, Dacinostat), colchicine, brefeldin A, diphenyleneiodonium chloride, any combination thereof or another agent identified herein. These treatment methods of the invention can also be used in combination with radiation treatment or other conventional cancer therapy.

This application claims priority under 35 U.S.C. §119(e) to U.S. Ser. No. 61/470,958, filed Apr. 1, 2011, which is incorporated herein by reference in its entirety.

### FIELD OF THE INVENTION

The discovery of clonally pure populations of serosal cancer stem cells (CSCs) as well as methods of producing CSCs, culturing the CSCs and using them in screening assays, has lead to the development of methods of treating serosal and ovarian cancers by targeting removal or inhibition of the glycocalyx coat surrounding such cells, and includes combination therapies using particular chemotherapeutics in conjunction with glycocalyx inhibitors, as well as the same new chemotherapy treatments without targeting the glycocalyx, where the chemotherapeutic agent is any one of LBH-589 (Panobinostat), NVP-AUY922, LAQ824 (NVP-LAQ824, Dacinostat), colchicine, brefeldin A, diphenyleneiodonium chloride, any combination thereof or another agent identified herein. These treatment methods of the invention can also be used in combination with radiation treatment or other conventional cancer therapy.

### BACKGROUND OF THE INVENTION

The cancer stem cell (CSC) hypothesis suggests that in cancer, either normal tissue stem cells become malignant or more differentiated tissue can be transformed and develop stem cell characteristics. Human CSCs are generally defined as a “rare” population of malignant cells that can undergo unlimited self-renewal with symmetric division capacity. These “tumor initiating cells” or cancer stem cells can regenerate all the components of the original tumor when serially transplanted.

The concept of cancer stem cells has had a major impact on our understanding of how to treat cancer. Unfortunately, unless CSCs can be eradicated, they may proliferate again and generate the cancer, leading to relapse. CSCs are thought to be particularly resistant to chemotherapy and radiation, making them particularly difficult to eliminate even with treatment that can efficiently destroy the bulk of the tumor and produce remission.

The CSC hypothesis depends on prospective purification of cells with tumor-initiating capacity, irrespective of frequency. The cancer stem cell hypothesis recognizes that the incidence of CSCs relative to more differentiated tumor cells can vary markedly from 0.001% to 100% depending on tumor type, stage of tumor development (e.g., metastatic vs. non-metastatic), or if studies were done on tumor cell lines selected from primary tumors, with high CSC content in the first place.

A number of in vitro assays, such as cloning in semi-solid medium, oncospheroid formation, limiting-dilution serial recloning, stromal colony formation, have been developed for CSCs. However, in vitro CSC assays are limited by the problem of an unknown and probably variable “plating efficiency” dependent on provision of, e.g., the appropriate combination and concentration of growth factors, morphogens and/or interactive niche components. The current “gold standard” for human CSCs is the tumor initiating limiting-dilution assay in immuno-deficient mice (Nude, SCID or NOD-SCID), however these recipients have innate immune resistance (Natural Killer (NK), macrophage). Furthermore, any in vivo assay has a “seeding efficiency” depending how efficient the cells are in localizing to their correct “niche.” If CSCs are injected into non-orthotopic sites (e.g., subcutaneously) lacking the appropriate “niche” or microenvironment (mesenchymal, endothelial), their numbers may be underestimated due to death or terminal differentiation. If injected intravenously, e.g., in metastatic models, the ability of CSCs to egress the vasculature and find appropriate niches may be determined by variable expression of homing receptors (e.g., integrins) and chemokine receptors (e.g., CXCR4), independent of the stem cell status of the cell. If the CSC is dependent on paracrine stimulation by growth factors or morphogens (e.g., IL-6, GM-CSF, M-CSF, IL-3 HGF), species specificity may exist. The existence of transit amplifying progenitor populations has been established in most tissues and such populations can generate billions of differentiated cells. Consequently, a primary in vivo assay for tumor development is not apriori a CSC assay unless re-passaging capacity can be demonstrated.

Ovarian cancer ranks fifth in cancer deaths among women and causes more deaths than any other gynecologic malignancy. It is estimated that in the United States 22,430 new cases will be diagnosed each year with 15,280 deaths [Jemal, 2008]. Ovarian carcinoma remains enigmatic in at least two important respects. First, the histological region of origin for this cancer remains obscure and second, an identifiable premalignant lesion that is generally recognized by cancer pathologists is yet to be defined. The majority (80%) of patients present with advanced stage disease with cancer cells throughout the abdominal cavity, leading directly to the high mortality (5 year survival rates 15-45%). In contrast, the survival rate for early stage disease, with malignancy confined to the ovary, is ˜95%. Given the discrepancy in survival outcomes between early- and late-stage diseases, strategies that would allow for the detection of ovarian cancer in its early stages would hold promise to significantly improve survival. Unfortunately, current screening methods for the detection of early stage ovarian cancer are inadequate.

The median overall survival for patients with advanced ovarian cancer has improved from approximately 1 year in 1975 to currently in excess of 3 years and for subsets having optimally debulked disease and treatment with taxane- and platinum-base combination chemotherapy, survival now exceeds 5 years [Ozols; Markman, 2003]. However the disease course is one of remission and relapse requiring intermittent re-treatment. Understanding the biology of CSCs and the mechanism by which such cells survive multiple rounds of chemotherapy to metastasize and regenerate tumors is important in the quest to find early stage detection methods and to eradicate ovarian cancer.

Opportunities to improve both overall survival and quality of life would include the development of novel therapies specifically designed to target the ovarian CSCs or other serosal CSCs. Eradicating cancer stem cells as well as differentiated cancer cells might increase the efficiency of therapy for ovarian or other serosal cancers, including metastatic serosal cancer.

The presence of cancer cells in effusions within the serosal (peritoneal, pleural, and pericardial) cavities is a clinical manifestation of advanced stage cancer and is associated with poor survival. Tumor cells in effusions most frequently originate from primary carcinomas of the ovary, breast, and lung, and from malignant mesothelioma, a native tumor of this anatomic site [Di Maria, 2007; Davidson, 2007]. Unlike the majority of solid tumors, particularly at the primary site, cancer cells in effusions are not amenable to surgical removal and failure in their eradication is one of the main causes of treatment failure [Davidson, 2007].

Formation of tumor spheroids (also referred to as oncospheroids) is a mechanism for tumor cells to adapt to grow in exudative fluids. Tumor spheroids are found in pleural, pericardial effusions and ascites samples from patients with serosal cancers. The pathophysiological relevance of tumor spheroids is best illustrated in ovarian cancer since a significant proportion of cancer cells in peritoneal ascites exist as spheroids. Advances in cancer therapy will depend on identification of novel therapeutic agents that can target CSCs that exists as individual entities or as these multicellular spheroids. Furthermore, screening systems will allow development of compounds toxic to both cycling stem cells and CSCs in a quiescent GO state.

While there have been some recent reports of isolation of subpopulations of cells from ovarian cancer that appeared to be enriched for cells capable of initiating tumors when transplanted into immunodeficient mice [Szotek, 2006; Zhang, 2008; Bapat, 2005], there have been no reports of clonally pure cells that can be maintained in their stem cell state in a tissue culture system. The lack of an in vitro system to maintain and expand clonally pure cells without differentiation has hindered the gene expression profiling and proteomics analysis of serosal cancer stem cells. Furthermore, lack of an in vitro culture system for CSC expansion has slowed down the development of high throughput drug screenings with potential to identify novel compounds that specifically target CSCs.

### SUMMARY OF THE INVENTION

As described in WO2011/057034 (to Moore et al.), the identification and isolation of clonally pure serosal cancer stem cells lead to the new insights for treating serosal cancer. In this application, the present invention is directed to methods of treating serosal cancer in a patient undergoing chemotherapy by administering a hyaluronan synthase inhibitor, a hyaluronidase, a collagenase, or a combination thereof, for a time and in an amount to augment the chemotherapy, to improve patient quality of life, to increase patient survival time and/or to cause remission of symptoms, wherein the chemotherapy comprises administering to the patient a therapeutically-effective amount of a compound selected from the group consisting LBH-589 (Panobinostat), NVP-AUY922, LAQ824 (NVP-LAQ824, Dacinostat), colchicine, brefeldin A and diphenyleneiodonium chloride, or a combination thereof. The hyaluronan synthase inhibitor, hyaluronidase and collagenase target degradation or inhibition of the glycocalyx and can be PEGylated or otherwise modified to increase their in vivo half life. In certain embodiments, the serosal cancer is ovarian cancer or a metastasis of ovarian cancer.

Administering the chemotherapy of the invention with the hyaluronan synthase inhibitor, hyaluronidase, collagenase, or combination thereof, can be done concurrently, or one after the other. In some embodiments, it may be preferable to begin degrading or inhibiting the glycocalyx before starting the particular chemotherapy, which means that the former can begin anywhere from a few days to a few weeks before the chemotherapy and overlap therewith or remain concurrent therewith for the duration of the chemotherapy.

In another aspect, the invention provides a method for treating serosal cancer in a patient by administering a therapeutically-effective amount of a compound selected from the group consisting LBH-589 (Panobinostat), NVP-AUY922, LAQ824 (NVP-LAQ824, Dacinostat), colchicine, brefeldin A, diphenyleneiodonium chloride and combinations thereof, to the patient. In certain embodiments, the serosal cancer is ovarian cancer or a metastasis of ovarian cancer.

In embodiments of both of the foregoing methods, the methods of the invention can be used in a patient before or after radiation treatments.

Yet a further aspect of the invention relates to a method to inhibit cancer stem cell self-renewal or formation by administering an inhibitor of glycocalyx formation or an agent that degrades glycocalyx for a time and in an amount to a patient to inhibit glycocalyx formation or degrade the glycocalyx of CSC in the patient and to thereby inhibit self-renewal or formation of said CSC, or to cause differentiation of the CSC and make them susceptible to killing, to prevent the catenae from undergoing spheroid formation, or any combination thereof, wherein the patient is also administered an effective amount of a compound selected from the group consisting LBH-589 (Panobinostat), NVP-AUY922, LAQ824 (NVP-LAQ824, Dacinostat), colchicine, brefeldin A, diphenyleneiodonium chloride or a combination thereof. The inhibitor or agent can be PEGylated or otherwise modified to increase its in vivo half life. In certain embodiments, the patient can also be administered one or more radiation treatments. In certain embodiments, the serosal cancer is ovarian cancer or a metastasis of ovarian cancer.

Clonally pure, serosal cancer stem cells are a self-renewing population of cells which comprise symmetrically dividing, free-floating chains of cells with from about three to four (3-4) to about seventy-two (72) cells, or more. The chains are surrounded by a glycocalyx of hyaluronan, collagen and other extracellular components. These cells are E-cadherin negative, have increased engraftment potential relative to serosal epithelial tumor cells and have at least 50% recloning capacity in vitro. In certain embodiments, the serosal cells are ovarian cells. These free floating chains are termed catenae or serosal cancer stem cells.

There are several methods to use the catena/spheroid system described herein to screen a test compound for anti-proliferative effects. For example, one can (a) culture any one of dissociated serosal catena cells, dissociated serosal spheroid cells or dissociated serosal cancer adherent cells, all of which cells are capable of fluorescence or luminescence; (b) contact the cells with a test compound; (c) detect whether the cells proliferate in response by detecting the fluorescence or luminescence emitted by the cultures; and (d) determine whether the test compound has inhibited proliferation of the catenae, spheroids or adherent cells. These methods may also include determining whether the test compound differentially inhibits proliferation of the catenae relative to the spheroids or adherent cells. Additionally, these methods can be adapted to screen a compound for its morphological effects on serosal cancer stem cells by having step (c) be detecting morphological changes (e.g., such as changes from catena to spheroid, spheroid to catena, catena to epithelial monolayer, catena to mesenchymal monolayer, spheroid to epithelial monolayer, spheroid to mesenchymal monolayer, or alterations in cell morphological shape, arrest at particular cell cycle stages, and the like). These methods can be readily adapted for high throughput screening (HTS) by growing the cells in 384- or 1536-well plates, for example, and conducting the assays using robotics systems for manipulating reagents, and collecting and analyzing the data. Such systems are known in the art.

In conducting screening assays with test compounds it was discovered that the sensitivity of the cells, in many but not all instances, depended on the presence of an established glycocalyx on the catenae and spheroids. Accordingly, if test compounds were added immediately or soon after seeding the cells (typically within one day), the cells were sensitive to the compound. However, if compounds were added several days later (typically 3-7 days), the glycocalyx had sufficient time to reestablish, and the cells became increasingly more resistant to the compound. In some cases, that resistance could be several orders of magnitude more than the compounds most sensitive effect on the cells. This effect was reversible if the glycocalyx was removed, thus rendering the cells once again sensitive to the compound. The acquired drug resistance overtime suggests that it is related to the resynthesis and organization of the pericellular glycocalyx. Hence, the glycocalyx may present a selective barrier to compounds depending on their chemical properties (size, polarity, hydrophobicity, diffusion). These observations lead to additional screening methodology and new methods of treating serosal cancer, the latter of which is the subjection of the present invention.

To find a compound with anti-proliferative or morphological effects, this screening method comprises (a) dissociating serosal catenae and preparing a homogenous population of single cells; (b) seeding and culturing those cells for a time and under conditions to produce catenae with an established glycocalyx coat; (c) contacting the cultures with at least one test compound for a time that would be sufficient to allow untreated cultures to proliferate without reaching confluency, i.e., the cultures should remain subconfluent during the course of the screening assay); and (d) determining whether the test compound inhibits proliferation of the catenae or alters morphology of the catenae in the treated culture. In some formats, the test compound(s) is added to the culture on day three, four, five, six or seven day post seeding, and more preferably on day five or six. In a variation on this method, following step (b) but prior to step (c), the culture can be incubated for a time and with an amount of a hyaluronidase, a collagenase or both, sufficient to remove or disrupt the glycocalyx coat of said catenae. Such treatments are typically done for about 5-30 minutes at 37° C., and preferably for about 10 minutes. These enzymes do not need to be removed for the duration of the remainder of the assay. Modified and PEGylated versions of the enzymes can also be used in the methods of the invention. These assays can also be readily adapted to an HTS format as above. To determine whether a test compound(s) affects proliferation, the cells can be counted manually with or without staining or a fluorescent signal, a luminescent signal or absorbance measured. Because the catenae exist in suspension, detection methods need to be adapted accordingly and can be done by those of skill in the art. One detection method is using alamarBlue® staining, followed by measuring fluorescence or absorbance of the culture which is proportional to the live cells present in the culture and is independent of whether the cells are adherent or in suspension.

A similar assay system for serosal spheroids is also disclosed. For spheroids, the dissociated cells are cultured for a time and under conditions to produce spheroids of sufficient number and size with an established glycocalyx coat. Because spheroids are large aggregates of many cells, it takes longer to reestablish the coat than it does for catenae. The time frame for spheroids is typically from about 8 to about 14 days, so that adding test compounds is done in that time frame, and preferably at 11 days post seeding.

The extensive characterization of the catenae lead to the discovery of multiple ways to identify catenae and to follow their fate, including by identification of specific surface antigens, catena gene signatures, surfaceome-related catena gene signatures, surfaceome-related catena protein signatures, miRNA-related catena signatures, catena cluster-defining gene signatures, exosomal catena protein signatures, secretome catena protein signatures, glycocalyx signatures, activated phosphoprotein expression, and identification of a low molecular weight complex of hyaluronan and collagen that binds to an anti-COL1A2 antibody. These properties have lead to a variety of methods to identify and/or monitor for the presence of serosal cancer stem cells in a patient sample and provides the ability for personalized medicine approaches to serosal cancer therapy, including the ability to alter a therapeutic regimen of the invention in response to the presence (or appearance) of serosal cancer stem cells.

These methods can be performed with serosal fluid, ascites, blood or tumor tissue from a mammal and using a variety of detection techniques including without limitation detecting the nucleic acids in these assays or determining expression levels thereof by microarray analysis, by an RNA or DNA sequencing technique, by RT-PCR or by Q-RT-PCR. Protein detection methods include but are not limited to mass spectrometry, Western blotting, antibody binding with FACS and other techniques with in the ken of the skilled artisan or later developed techniques.

Identifying and/or monitoring serosal cancer stem cells allows development of additional methods of the invention including methods to monitor efficacy of a cancer therapy regimen of the invention, to categorize patients for use of the chemotherapeutics of the invention, to monitor drug efficacy, to predict a patient response to a cancer therapy regimen of the invention in a serosal cancer patient by, for example, periodically performing one or more of these methods using a sample from a patient and correlating the results with the status of the patient and thereby monitoring efficacy of the cancer therapy regimen, categorizing the patient for appropriate therapy, monitoring drug efficacy or predicting a patient response to a cancer therapy regimen of the invention.

Hence, a still further aspect of the invention is directed to methods to treat a serosal cancer which comprises (a) administering an anticancer regimen in accordance with the invention (using the aforementioned chemotherapeutics) to a serosal cancer patient; (b) periodically monitoring the results from one or more methods to assess the catenae or spheroids using samples from the patient, and (c) altering the treatment regimen as needed and as consistent or predicted by the results.

Finally, the methods, cells, nucleic acids, vectors, proteins or genes described herein as mammalian include or can be human, murine, porcine, bovine or ovine mammals as applicable.

### BRIEF DESCRIPTION OF THE DRAWINGS

FIG. 1 present photographs of and a schematic representation of spheroid and catena formation. Ovcar3-GTL sphere-forming cells (red) pile up on mesenchymal monolayers (white) [stage 1-2], and form organized spheroids by budding [stage 3]. Catenae (blue) are observed inside [stage 4] or migrating out of developing spheroids [stage 5]. Developed spheroids detach from monolayers and continue to grow in suspension [stage 6] where more catenae are extruded into suspension.

FIG. 2 graphically illustrates a model of the catena-spheroid concept.

FIG. 3 is a bar graph showing the amount of CA125 (MUC16) secreted into the culture medium by subconfluent Ovcar3-GTL epithelial monolayers and catena as measured by ELISA.

FIG. 4 displays photographs of a particle exclusion assay using RBCs for (a) mechanically dissociated Ovcar3-GTL catenae and (b) hyaluronidase treated Ovcar3-GTL catenae.

### DETAILED DESCRIPTION OF THE INVENTION

### 1. Overview

The present application describes a clonally pure population of serosal cancer stem cells (CSCs), and methods of preparing and culturing these CSCs. With the availability of pure CSCs, extensive characterization of the cells was possible and lead to the elucidation of cell markers, morphology of the cells, identification of specifically expressed genes, identification of surfaceome markers, secretome markers, and from this information, target pathways for development of therapeutics and new treatment regimens. Purified CSCs are obtained as free-floating chains of cells, which are termed herein as catenae (plural; catena in the singular), with the capacity to self-renew and to differentiate. In addition to the serosal catenae, this application describes purified serosal spheroids and methods of isolating these cellular entities, allowing similar characterization studies of the spheroids at the molecular level.

The serosal cavity is a closed body cavity that includes and encloses the peritoneal, pleural, and pericardial cavities of the body, is fluid filled (serosal fluid) and is bounded by the serous membrane. Serosal cancers include the primary cancers that arise within the serosal cavity and secondary cancers that arise by metastasis of other cancer cells into the serosal cavity. Major serosal cancers at different serosal sites include those in (1) pleural effusions, namely mesothelioma, bronchogenic lung cancer, breast cancer, bladder cancer, ovarian cancer, fallopian tube cancer, cervical cancer and sarcoma; (2) peritoneal effusions, namely ovarian cancer, fallopian tube cancer, gastric cancer, pancreatic cancer, colon cancer, renal cancer and bladder cancer; and (3) pericardial effusions, namely mesothelioma, bronchogenic lung cancer, breast cancer, bladder cancer, ovarian cancer, fallopian tube cancer, cervical cancer and sarcoma. The list is not exhaustive, and any other cancer that metastasizes to any serosal cavity and forms tumors can be considered as a “serosal cancer.”

### 2. Miscellaneous Definitions

Serosal cells are any cells originating from or found within the serosal cavity or forming or attaching to the serous membrane, and include, but are not limited to, ovarian, endothelial, stomach, intestinal, anal, pancreatic, liver, lung and heart cells.

As used herein, NSG and NSG mice mean the NOD scid gamma (NSG) mice, or an equivalent, available from The Jackson Laboratory and which are the NOD.Cg-Prkdc^{scid} Il2rg^{tm1Wj1}/SzJ JAX® Mice strain. The NOG strain of mice are similar to NSG mice but have a truncated IL-2 receptor gamma chain rather than a complete null allele of the NSG mice.

As used herein, “chemotherapy” includes any form of cancer therapy in which one or more drugs is administered to a cancer patient for any and all cancer-related purposes, including without limitation, cytotoxic agents that inhibit or kill tumor cells (or other malignant cells) and cancer stem cells as well as agents that act in a cytostatic manner on such cells. Such drugs include, but are not limited to, small molecules, antibodies, proteins, nucleic acids, target pathway inhibitors and the like. For the avoidance of doubt, chemotherapy, as used herein, also includes pathway inhibitor therapy such as occurs when a subject has a genetic mutation in a specific gene and is administered a therapeutic agent targeted at that gene or the metabolic or regulatory pathway of which that gene forms part.

The abbreviations “ip” and “i.p.” are used interchangeably for intraperitoneal or intraperitoneally.

As used herein, ‘PEGylated” refers to a polyethylene glycol moiety (PEG) attached to a protein or other molecule of interest. PEGylation refers to the process of attaching a PEG to a protein or other molecule. Methodology for such modification is known in the art.

As used herein, the terms “therapeutically-effective amount” and “effective amount” are used interchangeably to refer to an amount of a drug or therapeutic agent that is sufficient to result in the prevention of the development, recurrence, or onset of cancer stem cells or cancer and one or more symptoms thereof, to enhance or improve the prophylactic effect(s) of another therapy, to reduce the severity and duration of cancer, to ameliorate one or more symptoms of cancer, to prevent the advancement of cancer, to cause regression of cancer, and/or to enhance or improve the therapeutic effect(s) of additional anticancer treatment(s).

### 3. Catenae

Clonally pure serosal CSCs are self-renewing serosal cells capable of differentiation and by this criterion meet the definition of stem cells. The CSCs comprise free-floating chains of cells having anywhere from three to four cells per chain to about seventy-two (72) cells, but this is not a precise upper bound as longer catena are occasionally observed. The catenae are surrounded by a glycocalyx comprising hyaluronan and resist attachment to tissue culture plates. As described in the methods of the present invention, catenae can be propagated in suspension cultures indefinitely. Each catena is clonal and cell division takes place symmetrically along the same axis, with occasional branching being observed. The capacity for symmetric division is independent of a cell's position in the chain, meaning that cells at the end and the middle of divide symmetrically and independently along the chain axis. This capacity to divide and propagate in culture establishes that the catena cells are self-renewing.

The cells are attached to each other via tight junctions which stain positively for ZO-1 but are negative for the presence of E-cadherin. Time lapse photography has shown that catenae do not fuse with each other but appear to repel each other.

When assessed in vitro, the catenae show at least 50% serial recloning capacity in limiting dilution assays. The individual catena cells have substantially increased in vivo engraftment potential relative to serosal epithelial tumor cells. Under appropriate conditions one or two catena cells can lead to engraftment of a tumor in a mouse cancer model. For example, in vivo engraftment is 50-100% in certain mice models (NSG mice) implanted subcutaneously with single catena cells in Matrigel. The catena engraft greater than 10,000 fold better over epithelial monolayers. This ability to form tumors after in vivo transplantation establishes that catenae have differentiation potential. Moreover, the tumors formed have similar morphology to those from which the cells were originally derived.

Similarly, catenae have the capacity to generate epithelial and mesenchymal monolayers in vitro under the appropriate conditions. It has been discovered that removing the glycocalyx (e.g., by hyaluronidase treatment) causes catenae to stop growing in suspension culture, settle onto tissue culture plates and begin to differentiate into mixed cultures of epithelial and mesenchymal cells.

Catenae grown in culture will continue to produce catenae, i.e., catenae are capable of serial passage in culture as non-attached cells. However, under appropriate conditions, such as when cultures become saturated, the catenae can round up and form spheroids. This rolling up action may provide a physical barrier means to protect CSCs from adverse conditions as spheroids contain about 10-30% CSC.

Catenae can be produced from serosal epithelial cancer cells or serosal mesenchymal cancer cells (discussed in detail below). Epithelial cells have polarized morphology and are E-cadherin positive and vimentin negative. Mesenchymal cells show a spindle morphology and are E-cadherin negative and vimentin positive. Catenae cells are rounded, and like mesenchymal cells, are E-cadherin negative and vimentin positive.

The catena's glycocalyx coat of hyaluronan is a predominant morphological feature and can be removed by treatment with hyaluronidase. The glycocalyx extends up to approximately 20 μm around the catena cells. When the glycocalyx is present, catenae grow in suspension culture and do not interact with extracellular matrix component. When the glycocalyx is removed enzymatically, the catena cells attached to surfaces, and form filopodial extensions and exhibit multilineage differentiation potential. Mechanically-dissociated catena cells remain in suspension and proliferate rapidly to form free-floating chain.

Scanning electron microscopy (SEM) of catena cells have shown a variety of pericellular structures in addition to the glycoclayx, including microvilli, nanotubes, pseudopodia, antenna and filopodia. In some instances, microvilli have been observed all over the cells and in other instances they tend to be located at the cell junctions, suggesting a role in cell-to-cell adhesion. The nanotubes are a novel cellular feature of CSCs and appear involved in cell-to cell communication, possible allowing passage of biomolecules between cells. The pseudopodia, antenna and filopodial may play a role in formation of the nanotubes as well as allow surveillance of the environment for attachment surfaces and the presence of cytokines, growth factors and immune cells.

In addition, SEM has shown that the catena cells have surface blebs and structures that appear to erupt from the cell surface and release smaller particles. These erupting structures appear as either “volcanoes” or invaginated “craters.” The released particles are similar in appearance and size to the surface blebs and appear to be exosomes.

Transmission electron microscopy (TEM) shows that the catena cells have the undifferentiated cell morphology (high nucleus to cytoplasm ratio) typical of stem cells. TEM also allowed observation of the tight junctions between the cells and showed that intact functional mitochondria are present. Surface blebs were observed to be contiguous with the cell membrane and to contain ribosomes.

Having a clonally pure population of cells allowed molecular characterization of ovarian catenae (i.e., ovarian CSCs). Using gene expression, the gene signature for ovarian catena relative to ovarian mesenchymal monolayer cancer cells is shown in Table 5 of WO2011/057034. The gene signature has 26 upregulated genes and 69 down regulated genes, with hyaluronan synthase (HAS2) the most highly expressed gene in catenae/CSCs. The second most expressed gene was PDGFRA indicating a significant role for the PDGF pathway in catenae/CSCs.

Using differential miRNA expression analysis, it was discovered that the miR-200 family (miR-141, miR-200a, miR-200b, miR-200c and miR-429) and the Let-7 family miRNAs were significantly down-regulated in the ovarian catenae compared to ovarian epithelial monolayers. Further, hsa-miR-23b and hsa-miR-27b were significantly down regulated in ovarian catena compared to ovarian mesenchymal monolayers.

Using a receptor tyrosine kinase (RTK) phosphorylation assay, it was shown that ovarian catenae cells and ovarian mesenchymal cancer cells have qualitatively similar phospho-RTK profiles.

Using cell surface marker analysis with commercially available antibodies and FACS, ovarian catenae are positive for the markers CD49f (a6-integrin), CD90, GM2 and CD166 and negative for the markers EpCam (CD326), Mucl6(CA125) and CD44.

### 4. Spheroids

Serosal spheroids are large cellular structures composed of tens of thousands of cells were observed as entities that would not pass through a 40 μm filter. Spheroids may play a role in metastasis and tumor formation. Spheroids also self-renew in suspension cultures and have differentiation capacity. When assessed in vitro, spheroids have about a 10% serial recloning capacity in limiting dilution assays.

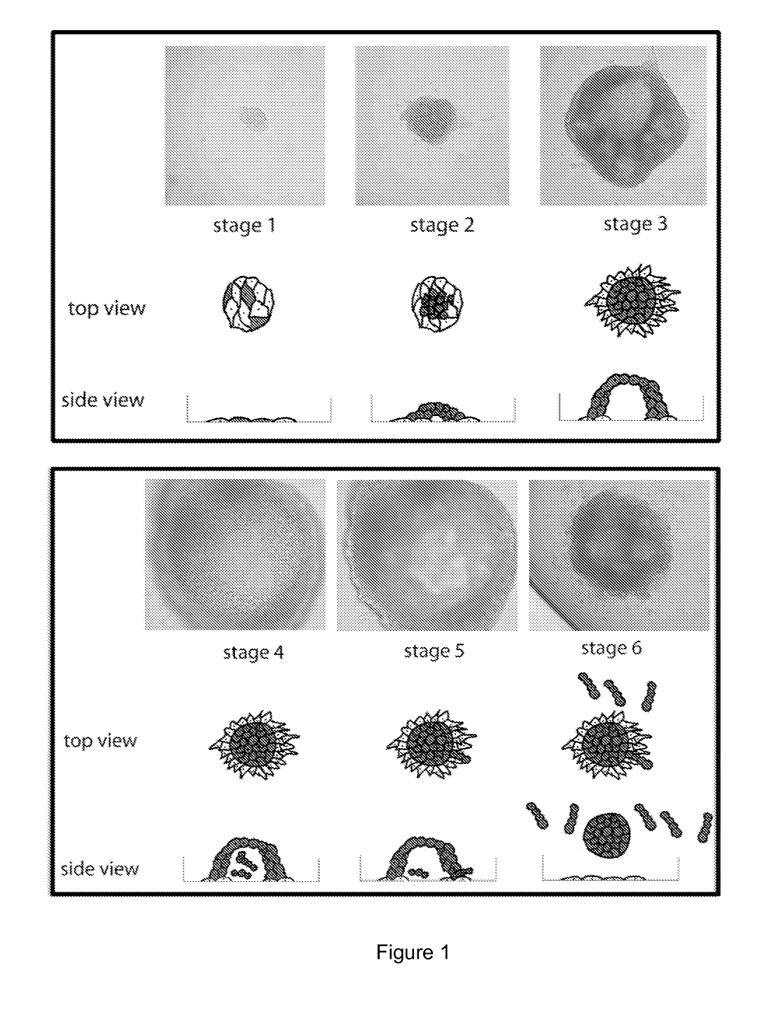

Spheroids developed from catenae by a process of “rolling up,” suggesting that during nutrient deprivation at confluent stages of cell culture, spheroids provide a protective environment for catenae survival. Additionally, cells can amass on attached mesenchymal monolayers and begin to form spheroids. This cell mass grows in the vertical direction relative to attachment surface, resembling “budding” from attached cells, and develops into spheroids with organized cystic structures. The spheroids eventually detach from attached monolayers and continue to rapidly proliferate in suspension while maintaining the sphere morphology. A schematic diagram of this process is shown in FIG. 1. Developing spheroids extrude fresh catenae into the suspension which in turn can proliferate rapidly to form new floating catenae.

### 5. Preparation of Catenae and Spheroids

Two principal methods for preparing catenae and spheroids are described herein. In one method, serosal epithelial or mesenchymal cancer cells are injected intraperitoneal (ip) into an animal tumor model (preferably mice), preferably with the addition of an inflammatory stimulus. After sufficient time to develop ascites and/or solid tumors, the ascites is harvested from ip tumor-bearing animals and separated into two or more size fractions, preferably two fractions. The smaller size fraction contains the catenae and single cells, typically leukocytes. The leukocytes can be readily removed and the remaining cells serially passaged in suspension culture to obtain a self-renewing population of clonal serosal catenae. The larger fraction includes the spheroids retained on the filter. These spheroids are collected and serially passaged in a suspension culture to obtain a self-renewing population of spheroids.

The source of the serosal epithelial cells can be from primary serosal cancer cells, or immortalized epithelial or mesenchymal serosal cancer cell lines. The primary cancer cells or cell lines can be from primary cancers or metastatic tumors. Preferably the serosal cancer cells are ovarian cancer cells.

As used herein, an animal tumor model is an animal capable of allowing tumor formation and is typically highly immunodeficient, i.e., lacking at least B cells and T cells and preferably also NK cells. For example, a preferred animal is a NOD-SCID ILR gamma (−/−) mouse (referred to herein as a “NSG” mouse) which lack B cells, T cells and NK cells. NOD-SCID mice lack B cells and T cells, and while useful, require injection of much greater more cell numbers to develop tumors.

Inflammatory stimuli include any agent, drug or factor (collectively referred to herein as inflammatory agents) that stimulate inflammation in an animal, and are preferably administered i.p. Inflammatory agents include, but are not limited to, lipidated oligonucleotides, thioglycollate; chemerin; macrophage migration-inducing chemokines such as chemokine (C-C motif) ligand 1 (CCL1), CCL2, CCL4, CCL7, CCL8, CCL12, CCL13, CCL15, CCL16, CCL23 and CCL25; macrophage activating chemokines such as CCL14; and various agents of bacterial origin including, brewer's thioglycollate broth (3%), BCG heat-killed (cell walls from M. bovis), pyran copolymer, C. parvum heat-killed whole cells, pyridine extract of C. parvum, detoxified endotoxin from Salmonella typhimurium; and sodium metaperiodate. The lipidated oligonucleotides are typically small oligomers of from about 8 to about 30 nucleotides and act in a sequence independent manner. The lipid moiety can be any convenient group such as myristate, palmitate and the like. Those of skill in the art can determine appropriate doses for administering inflammatory agents.

Size fractionation can be done by passing the ascites through one or more filters. Useful filter sizes range from about 20-60 μm, with larger sizes allowing more spheroids to pass through. A preferred filter size is 40 μm.

In another method, catenae and spheroids can be produced by in vitro culture techniques from immortalized serosal mesenchymal cancer cells. In this method, the mesenchymal cells are grown as monolayers, the culture supernatant is harvested and the suspension cells are pelleted by gentle centrifugation (e.g., at 300 g for 1-5 minutes). The pelleted cells are resuspended in fresh media (typically at one-tenth the previous culture density), transferred to fresh suspension culture flasks for growth. Repeating this cycle several times produces self-renewing populations of serosal catenae and spheroids. Typically the cells are grown until they reach a cell density of about 200,000 cells/mL or can be passaged weekly. Likewise, this process appears to remove an inhibitory factor produced by mesenchymal monolayers that prevents catenae and spheroid formation. These cultures can be size fractionated as above to separate the catenae from the spheroids.

The growth media for these methods is any convenient media supplemented with 10% fetal calf serum (FCS). Cells are generally grown at 37° C. with 5% CO₂. A preferred growth media for catenae is M5 with 10% FCS (Hyclone) and 1% P/S (Pen-Strep Solution at 10,000 U/mL penicillin G and 10 mg/mL streptomycin; Gemini Bio-Products), designated hereafter as M5-FCS. M5 media is DME:F12, 6 g/L HEPES and 2.2 g/L sodium bicarbonate. Catenae can also be grown in serum-free, protein-free media supplemented with insulin. One such preferred media is M5 with 1% P/S and 0.1 U/mL recombinant insulin. The insulin source should be the same as the cell source, i.e., if human catenae are being cultured, the serum free media is supplemented with recombinant human insulin, etc.

A preferred growth media for spheroids is ES media, and preferably supplemented mTeSR1 media [Ludwig et al. 2006].

### 6. Gene Signature and Other Methods to Identify CSCs

The gene expression information provided in Table 5 of WO2011/057034 may be used as diagnostic markers for the identification of the ovarian CSCs. For example, ascites or an ovarian tissue sample from a patient may be assayed using a gene microarray, RNA sequencing, RT-PCR, Q-RT-PCR, 454 deep sequencing, or other methods known to those of skill in the art, to determine the expression levels of one or more of the genes in Table 5 of WO2011/057034. These levels may be compared to the expression levels found in normal tissue, ovarian mesenchymal cancer cells or ovarian epithelial cancer cells. Expression levels can also be used as markers for the monitoring of disease state, disease progression, especially metastasis, or as markers to evaluate the effects of a candidate drug or agent on a cell or in a patient. Assays which monitor the expression of a particular genetic marker or markers can utilize any available means of monitoring for changes in the expression level of the relevant genes. As used herein, an agent is said to modulate the expression of a gene if it is capable of up- or down-regulating expression of mRNA levels of that gene in a cell.

The present invention provides the following methods to identify and/or monitor for the presence of serosal cancer stem cells in a patient sample.

With respect to the catena surfaceome, this invention provides a method to identify and/or monitor for the presence of serosal cancer stem cells in a patient sample which comprises (a) obtaining a cellular sample from a patient; (b) depleting the sample of leukocytes; (c) reacting the sample with a panel of detectable surface antigen antibodies; (d) sorting the reacted cells into single- or multi-cell samples; and (e) detecting whether any of said single- or multi-cell samples are positive for the presence of CD49f, CD90, CD 166, PDGFRA, and GM2 proteins and negative for the presence of CD34, CD133, MUC16 and EPCAM proteins, wherein the presence and absence of said proteins identifies the reacted cells as containing serosal cancer stem cells or identifies a single cell as a serosal cancer stem cell.

Sorting cells, including to the single cell level, can be done, for example, by fluorescent activated cell sorting (FACS) using appropriately distinguishably labeled antibodies.

Alternatively, surfacesome characteristics can be used in a method to identify and/or monitor for the presence of serosal cancer stem cells in a patient sample which comprises (a) obtaining a cellular sample from a patient; (b) depleting the sample of leukocytes; (c) extracting RNA from the remainder of the sample; (d) analyzing the RNA for expression levels of a human mRNA transcriptome; and (e) identifying samples having a surfaceome-related catena gene signature as those which have upregulated HAS2 and PDGFRA, downregulated MUC16 and EPCAM and have upregulated at least 7 additional genes listed in Table 11 of WO2011/057034, wherein having those characteristics indicates the patient sample contains serosal cancer stem cells.

Likewise, the surfaceome properties can be used in a method to identify and/or monitor for the presence of serosal cancer stem cells in a patient sample which comprises (a) obtaining an integral membrane protein fraction from a cellular sample of a patient, wherein the cellular sample has optionally been depleted of leukocytes; (b) analyzing the protein content of said membrane fraction by mass spectrometry; (c) identifying samples having a surfaceome-related catena protein signature as those samples in which the spectral data indicate the presence of at least 40 proteins listed in Table 16 of WO2011/057034, wherein presence of those proteins indicates the patient sample contains serosal cancer stem cells. One method to prepare an integral membrane fraction is to isolate cells and use phase partitioning process with Triton X-114 to prepare a detergent soluble fraction that can be analyzed by mass spectrometry.

Based on the information from the catena miRNAs that have been characterized, the present invention provides a method to identify and/or monitor for the presence of serosal cancer stem cells in a patient sample which comprises (a) obtaining a cellular sample from a patient; (b) depleting the sample of leukocytes; (c) extracting RNA from the remainder of the sample; (d) analyzing the RNA for expression levels of human miRNA; and (e) identifying samples having an miRNA-related catena signature as those which have downregulated let-7 and 200 families of miRNA, downregulated hsa-miR-23b and hsa-miR-27b, and have upregulated at least 4 additional miRNA listed in Table 8 of WO2011/057034, wherein having those characteristics indicates the patient sample contains serosal cancer stem cells.

Using analysis for the expression of all catena mRNA established a catena gene signature. Hence, another method to identify and/or monitor for the presence of serosal cancer stem cells in a patient sample which comprises (a) obtaining a cellular sample from a patient; (b) depleting the sample of leukocytes; (c) extracting RNA from the remainder of the sample; (d) analyzing the RNA for expression levels of a human mRNA transcriptome; and (e) identifying samples having a catena gene signature as those samples which have upregulated HAS2 and PDGFRA and have upregulated at least 5 additional genes listed in Table 5 of WO2011/057034, wherein having those characteristics indicates the patient sample contains serosal cancer stem cells. Another embodiment uses a catena cluster-defining gene signature and provides a method to identify and/or monitor for the presence of serosal cancer stem cells in a patient sample which comprises (a) obtaining a cellular sample from a patient; (b) optionally, depleting the sample of leukocytes; (c) extracting RNA from the remainder of the sample; (d) analyzing the RNA for expression levels of a human mRNA transcriptome; and (e) identifying samples having a catena cluster-defining gene signature as those samples which have upregulated at least six of the nine genes in LIST1 of Table 7 of WO2011/057034 and have upregulated at least 5 of the genes in LIST2 of Table 7 of WO2011/057034, wherein having a catena cluster-defining gene signature indicates the patient sample contains serosal cancer stem cells.

One can identify serosal cancer stem cells in a subject by the method which comprises (a) detecting the level of expression of ten or more genes from Table 5 of WO2011/057034 in a tissue sample, wherein increased or decreased expression of the genes in accordance with that table and relative to expression in serosal mesenchymal monolayer cells is indicative of the presence of serosal cancer stem cells.

The catena exosomes and secretomes are particularly useful for methods of identifying and/or monitoring serosal cancer stem cells. For example, the exosomal catena protein signature can be used in a method to identify and/or monitor for the presence of serosal cancer stem cells in a patient sample which comprises (a) obtaining isolated exosomes from a patient sample; (b) analyzing the protein content of said exosomes by mass spectrometry, by antibody binding or otherwise; (c) identifying samples having an exosomal catena protein signature as those samples in which the spectral data or other data indicate the presence of CD63, COL1A2 and at least 5 additional proteins listed in Table 1 of WO2011/0570343, wherein presence of said proteins indicates the patient sample contains serosal cancer stem cells.

Exosomal catena protein signature can be used in a method to identify and/or monitor for the presence of serosal cancer stem cells in a patient sample which comprises (a) obtaining isolated exosomes from a patient sample; (b) reacting said exosomes with one or more antibodies specific for CD63, COL1A2 and at least 5 additional proteins listed in Table 13 of WO2011/057034; and (c) identifying samples having an exosomal catena protein signature as those samples in which are positive for the presence of CD63, COL1A2 and at least 5 additional proteins listed in Table 13 of WO2011/057034, wherein presence of said proteins indicates the patient sample contains serosal cancer stem cells.

The secretome catena protein signature can be used in a method to identify and/or monitor for the presence of serosal cancer stem cells in a patient sample which comprises (a) obtaining a supernatant fraction from a patient sample from which cells, cellular debris and exosomes have been removed; (b) analyzing the protein content of said supernatant fraction by mass spectrometry; (c) identifying samples having a secretome catena protein signature as those samples in which the spectral data indicate the presence of at least 20 proteins listed in Table 15 of WO2011/057034, wherein presence of those proteins indicates the patient sample contains serosal cancer stem cells.

Still another embodiment uses a glycocalyx signature and provides a method to identify and/or monitor for the presence of serosal cancer stem cells in a patient sample which comprises (a) obtaining a supernatant fraction from a patient sample from which cells, cellular debris and exosomes have been removed; (b) analyzing the protein content of said supernatant fraction by mass spectrometry; (c) identifying samples having a glycocalyx signature as those samples in which the spectral data indicate the presence of at least 6 proteins found in glycocalyx as listed in Table G and the absensce of ELN, FN1 and at least 2 protein downregulated in catena as listed in Table G, wherein presence and absence of those proteins indicates the patient sample contains serosal cancer stem cells.

Based on phosphorylation of tyrosine kinase receptors (RTK), another embodiment of the invention is directed to a method to identify and/or monitor for the presence of serosal cancer stem cells in a patient sample which comprises (a) obtaining a cellular sample or a cell lysate from a cellular sample from a patient, wherein said sample has been depleted of leukocytes; (b) incubating said sample or said lysate with a panel of human tyrosine kinase receptor-specific antibodies and a pan-phosphotyrosine antibody; and (c) detecting whether said sample or lysate is positive for activated phosphoproteins selected from the group consisting of PDGFRA and at least 6 of the proteins selected from the group consisting of PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphAl, EphA3, EphA4, EphA7, EphB2, EphB4, and EphB6, wherein the detection of said activated phosphoproteins identifies the patient sample as containing serosal cancer stem cells.

Based on the composition and characterization of the glycocalyx, one can identify and/or monitor for the presence of serosal cancer stem cells in a patient sample by a method which comprises (a) obtaining a supernatant fraction from a patient sample from which cells and cellular debris have been removed; (b) reacting the sample with an anti-COL1A2 antibody; (c) detecting whether said antibody binds a low molecular weight complex of hyaluronan and collagen of less than 20,000 Daltons, wherein the detecting said complex indicates that said sample contains serosal cancer stem cells

The samples for the methods in this section can be mammalian serosal fluid, ascites, blood or tumor tissue. Preferably, the mammal is a human.

The various steps of detecting, determining, analyzing and the like can be conducted by methods known to those of skill in the art. For example, with the appropriate methods, detecting of a nucleic acid or determining expression levels can be accomplished by microarray analysis, by an RNA or DNA sequencing technique, by RT-PCR, by Q-RT-PCR and the like.

Further, the above methods form the basis of additional embodiments of the instant invention. For example, this invention provides a method to detect serosal cancer, to monitor efficacy of a cancer therapy regimen, to categorize patients for therapy, to monitor drug efficacy, to predict a patient response to a cancer therapy regimen in a serosal cancer patient which comprises (a) periodically performing one or more methods of the above methods (e.g., as set out in original claims 48-67) with samples from a patient and (b) correlating the results with the status of the patient to thereby detect serosal cancer, to monitor efficacy of a cancer therapy regimen, to categorize a patient for therapy, to monitor drug efficacy or to predict a patient response to a cancer therapy regimen.

PCR primer sets for identifying serosal CSCs by any one of the myriad of PCR amplification methods known in the art for DNA, RNA or both. Those of skill in the art can select the appropriate sequences to for the PCR primers from the known sequence of the human genome. PCR primers sets for mammalian genes include, but are not limited to, the following combinations (each combination being a PCR primer set for amplification and detection of the indicated genes within that set):

(a) CD49f, CD90, CD166, PDGFRA and GM2 genes;

(b) CD49f, CD90, CD166, PDGFRA, GM2, CD34, CD133, MUC16 and EPCAM genes;

(c) HAS2, PDGFRA and at least 10 of the upregulated genes listed in Table 11 of WO2011/057034;

(d) HAS2, PDGFRA, MUC16, EPCAM and at least 10 of the upregulated genes listed in Table 11 of WO2011/057034;

(e) the genes of at least 40 of the proteins listed in Table 16 of WO2011/057034;

(f) let-7 and 200 miRNA families, hsa-miR-23b and hsa-miR-27b, and at least 4 additional miRNAs listed in Table 8 of WO2011/057034;

(g) HAS2, PDGFRA and at least 5 additional genes listed in Table 5 of WO2011/057034;

(h) the nine genes in LIST1 of Table 7 and at least 5 genes in LIST2 of Table 7 of WO2011/057034;

(i) ten or more genes from Table 5 of WO2011/057034;

(j) CD63, COL1A2 and at least 5 additional genes for the proteins listed in Table 13 of WO2011/057034;

(k) the genes of at least 20 proteins listed in Table 15 of WO2011/057034;

(l) the genes of at least 6 glycocalyx proteins as listed in Table G;

(m) ELN, FN1, the genes of at least 6 glycocalyx proteins as listed in Table G, and the genes of at least 2 proteins listed as downregulated in Table G; and

(n) PDGFRA and the genes for at 6 of the proteins selected from the group consisting of PDGFRβ, EGFR, ERBB4, FGFR2, FGFR3, Insulin-R, IGF1R, DTK/TYRO3, MER/MERTK, MSPR/RON, Flt-3, c-rRET, ROR1, ROR2, Tie-1, Tie-2, TrkA/NTRK1, VEGFR3, EphAl, EphA3, EphA4, EphA7, EphB2, EphB4, and EphB6.

### 7. Drug Screening Methods

The drug screening methods described herein were used to identify chemotherapeutic agents for treating serosal cancers. For example, such methods include methods to screen a test compound for anti-proliferative effects by (a) culturing dissociated serosal catena or serosal spheroid cells that are detectable by fluorescence or luminescence; (b) contacting said catena or spheroids with a test compound; (c) detecting proliferation of said catena or spheroids by measuring the fluorescence or luminescence produced by the cultures relative to control cultures; and (d) determining if the test compound inhibits proliferation of said catena or spheroids.

Similarly, another method to screen a test compound for anti-proliferative effects on serosal cancer stem cells comprises (a) culturing dissociated serosal catena cells, dissociated serosal spheroid cells and dissociated serosal cancer adherent cells, each of which are detectable by fluorescence or luminescence, in parallel; (b) contacting said cells with said test compound; (c) detecting proliferation of catena, spheroids and adherent cells by measuring the fluorescence or luminescence produced by the cultures relative to control cultures; (d) determining if the test compound differentially inhibits proliferation of the catenae relative to spheroids and monolayers.

In these methods, cells are conveniently grown in multi-well plates such as 96-well, 384-well or 1536-well plates. The various manipulations to add media, seed the plates, add test compounds and score the results can be done manually or robotically on apparatus designed for this purpose. Similarly, the assay results can be determined manually, or can be adapted to automated or robotic analyzers. For detecting anti-proliferative effects, the fluorescent signal from the cell cultures can be at assessed at discreet time points or monitored continuously as is suitable for the assay.

Another embodiment provides methods to screen test compounds (or agents) for phenotypic or other effects on serosal catenae, spheroids and monolayers. These methods are conducted in a manner similar to the above assays to assess the anti-proliferative effects of test compounds, except for the detection method. In these embodiments, the detection method depends on the particular property being assessed and being distinctly detectable. For differentiation inhibitors, the detection method can assess whether catena cells fail to differentiate in culture upon exposure to the compound.

In conducting screening assays with test compounds it was discovered that the integrity of the glycocalyx can play an important role is drug sensitivity or resistance of the cells. While some compounds can readily penetrate the glycocalyx, others cannot. For the compounds used in chemotherapy which eventually cease to be efficacious in a patient, the knowledge that a drug or chemotherapeutic has lost effectiveness due to the possible renewed presence means that such drugs could maintain efficacy, and hence be used again, if the glycocalyx of the serosal cancer stem cells could be removed. This recognition created a need for another way to screen test compounds or drugs, know chemotherapeutics and the like for the ability to inhibit proliferation or alter the morphology of catena and spheroids under conditions where these cellular entities have of an established and/or substantial glycocalyx.

Accordingly, another embodiment provides a method to screen a test compound for anti-proliferative or morphological effects which comprises (a) dissociating serosal catenae and preparing a homogenous population of single cells; (b) seeding and culturing those cells for a time and under conditions to produce catenae with an established glycocalyx coat; (c) contacting the cultures with at least one test compound for a time that would be sufficient to allow untreated cultures to proliferate without reaching confluency, i.e., the cultures should remain subconfluent during the course of the screening assay); and (d) determining whether the test compound inhibits proliferation of the catenae or alters morphology of the catenae in the treated culture. In a preferred embodiment, the test compound(s) is added to the culture on day three, four, five, six or seven day post seeding, and more preferably on day five or six. In a variation on this method, following step (b) but prior to step (c), the culture can be incubated for a time and with an amount of a hyaluronidase, a collagenase or both, sufficient to remove or disrupt the glycocalyx coat of said catenae. Such treatments are typically done for about 5-30 minutes at 37° C., and preferably for about 10 minutes. These enzymes do not need to be removed for the duration of the remainder of the assay. Modified and PEGylated versions of the enzymes can also be used in the methods of the invention. These assays can also be readily adapted to an HTS format as above. To determine whether a test compound(s) effects proliferation the cells can be counted manually with or without staining or a fluorescent signal, a luminescent signal or absorbance measured. Because the catenae exist in suspension, detection methods need to be adapted accordingly and can be done by those of skill in the art. One preferred detection method is using alamarBlue® staining, followed by measuring fluorescence or absorbance of the culture which is proportional to the live cells present in the culture and is independent of whether the cells are adherent or in suspension.

A similar assay system for serosal spheroids is also provided. For spheroids, the dissociated cells are cultured for a time and under conditions to produce spheroids of sufficient number and size with an established glycocalyx coat. Because spheroids are large aggregates of many cells, it takes longer to reestablish the coat than it does for catenae. The time frame for spheroids is typically from about 8 to about 14 days, so that adding test compounds is done in that time frame, and preferably at 11 days post seeding.

Hence, these methods allow for screening compounds for their toxicity and their chemical properties against serosal (including ovarian) cancer stem cells (catenae) with their protective pericellular coat undisturbed and represent an in vitro system that is more relevant to the clinical setting than conventional screening methods. The in vivo and in vitro data suggest that catenae are ovarian cancer stem cells adapted to grow in suspension in ascites fluid and that glycocalyx formation, without be limited to a mechanism, might be necessary for growth and expansion of cancer stem cells in ascites fluid and to remain as cancer stem cells. The data also explains the resistance to therapy in advanced stage ovarian cancer with peritoneal metastasis and other serosal cancer types. Any compound identified as toxic to catena with intact pericellular coat in this screen is potentially useful in treatment of advanced stage ovarian cancer.

### 8. Treatment Methods

A. Targeting the Glycocalyx

The catena's glycocalyx coat of hyaluronan is a predominant morphological feature. Targeting this feature for removal, provides a method of treating serosal cancer, maintaining cancer in a manageable disease state, eradicating cancer stem cells after or during other standards of cancer care (e.g., in conjunction with chemotherapy or radiation treatment) as well as prolonging the time to relapse or metastasis.

Hyaluronan and/or other glycocalyx components may be targeted through a variety of paths including degradation of hyaluronan, prevention of hyaluronan binding to its receptors (for example: CD44, RHAMM), prevention of hyaluronan export or proteins that interact with hyaluronan (for example: Aggregan, Versican). Additionally, hyaluronan expression may be inhibited or reduced by targeting synthetic pathway components which produce hyaluronan by various techniques including RNAi or antisense or addition of enzyme inhibitors. Hyaluronan synthesis can be disrupted by inhibiting formation of parts of its chemical structure (for example: targeting the repeating disaccharide units or the glycosidic bonds). Further, inhibition of hyaluronan synthesis may be accomplished by targeting hyaluronan synthase (HAS) on a DNA, RNA, or protein level (e.g., enzymatic inhibitors). Examples HAS inhibitors include, but are not limited to, 4-methylumbelliferone (4-MU or MU), 4-methylesculetin (ME), brefeldin A, mannos, siRNA against hyaluronan synthase enzymes, antibodies against extracellular or intracellular domains of hyaluronan synthase enzymes, and hyaluronidase (bacterial or animal origin, natural or recombinant) as well as PEGylated or chemically modified derivatives of any of any of the foregoing (as appropriate).

Hyaluronan can be targeted for degradation or removal by antibodies, small molecules, enzymes or other means. Hyaluronan is most commonly degraded by hyaluronidase, a glycoprotein. Hyaluronidase has been recognized as having a potential therapeutic use in cancer. This enzyme or modifications that can be used in animals may be used here for the first time to selectively target serosal cancer stem cells. For example, ovarian cancer is commonly treated with standard therapies including surgery, chemotherapy, radiation, or a combination of these. Such treatment may include platinum based therapies, topotecan, oral etoposide, docetaxel, gemcitabine, 5-FU, leucovorin, liposomal doxorubicin.

Hence, such treatments can be supplemented with a course of treatment to remove or inhibit glycocalyx formation. For example, in one treatment regimen, the primary cancer is removed (by any means or treatment), followed by hyaluronidase treatment to eradicate any catenae or CSCs that are resistant or escape treatment. Hyaluronidase treatment can also be done concurrently with standard courses of cancer treatment. Further these two therapeutic modalities can be followed by additional rounds of standard therapy (e.g., chemo) if needed.

The invention contemplates methods of care that eradicate, disrupt morphology, force differentiation, or decrease the clonogenicity of the catena which include hyaluronidase treatment as part of the treatment.

Accordingly, the present invention is directed to methods of treating serosal cancer in a patient undergoing chemotherapy by administering a hyaluronan synthase inhibitor, a hyaluronidase, a collagenase, or a combination thereof, for a time and in an amount to augment the chemotherapy, to improve patient quality of life, to increase patient survival time and/or to cause remission of symptoms, wherein the chemotherapy comprises administering to the patient a therapeutically-effective amount of a compound selected from the group consisting LBH-589 (Panobinostat), NVP-AUY922, LAQ824 (NVP-LAQ824, Dacinostat), colchicine, brefeldin A and diphenyleneiodonium chloride, or a combination thereof. These particular chemotherapeutic agents used in the methods of the invention were identified as useful for treating serosal cancers in accordance with the screening methods disclosed herein.

Administering the chemotherapy of the invention with the hyaluronan synthase inhibitor, hyaluronidase, collagenase, or combination thereof, can be done concurrently, or one after the other, in either order. In some embodiments, it may be preferable to begin degrading or inhibiting the glycocalyx before starting the particular chemotherapy, which means that the former can begin anywhere from a few days to a few weeks before the chemotherapy and overlap therewith for the duration of the chemotherapy.

In another aspect, the invention provides a method for treating serosal cancer in a patient by administering a therapeutically-effective amount of a compound selected from the group consisting LBH-589 (Panobinostat), NVP-AUY922, LAQ824 (NVP-LAQ824, Dacinostat), colchicine, brefeldin A, diphenyleneiodonium chloride and combinations thereof, to the patient.

The above methods lead to remission of cancer symptoms, e.g., including tumor regression, less bloating or less ascites formation. These methods also inhibit cancer stem cell self-renewal and/or formation in a patient, without being bound to a mechanism, by inhibiting glycocalyx formation by said CSC which thereby inhibits self-renewal and causes differentiation of the CSC. This differentiation may then make the cells again susceptible to standard cancer treatment regimens known in the art.

Serosal cancers, include but are not limited to, ovarian cancer and any cancer that appears in the serosal cavity, whether of primary or secondary (e.g., metastatic) origin.

Enzymes that catalyze hyaluronan breakdown (degrade hyaluronic acid) include the hyaluronidases (e.g., EC 3.2.1.35). Humans have six associated genes, including HYAL1, HYAL2, HYAL3, HYAL4, MGEA5 and PH-20/SPAM1. Any hyaluronidase can be used in the invention. A preferred hyaluronidase for use in the present invention is recombinant human hyaluronidase Hylenex (Halozyme Therapeutics) derived from the gene PH20. Pegylated PH20 hyaluronidase is also useful.

Hyaluronidase can be of human, other animal or bacterial origin, as well as artificially made (recombinant/synthetic). It may be modified (pegylation, addition of a transporter of oligomers, other commonly known ways to modify an enzyme) and can be provided in any formulation that delivers an effective dose to a patient. Methods of determining dosages and formulating chemotherapeutics are known to those of skill in the art.

In another aspect, the invention is directed to a method to inhibit cancer stem cell self-renewal or formation in a patient which comprises administering an inhibitor of glycocalyx formation or an agent that degrades glycocalyx for a time and in an amount to said patient to inhibit glycocalyx formation or degrade the glycocalyx of CSC in the patient and thereby inhibit self-renewal or formation of said CSC, to cause differentiation of the CSC, to make the CSC susceptible to killing by other chemotherapeutic regimens, or to prevent catena from undergoing spheroid formation.

The inhibitors and enzymes used in the methods of the invention can be provided as pharmaceutical compositions for a variety of delivery methods, including but not limited to, intraperitoneal or intraserosal delivery in the form of injectable sterile solutions, suspensions or other convenient preparation. Intraserosal administration includes intrapleural, intrapercardial and intraperitoneal injections. Intraperitoneal delivery is particularly useful.

When administered orally, the inhibitors and enzymes can be, for example, in the form of pills, tablets, coated tablets, capsules, granules or elixirs. Administration can also be carried out rectally, for example in the form of suppositories, or parentally, for example intravenously, intramuscularly, intrathecally or subcutaneously, in the form of injectable sterile solutions or suspensions, or topically, for example in the form of solutions or transdermal patches, or in other ways, for example in the form of aerosols or nasal sprays. Depending on the nature of the administration, the pharmaceutical compositions may further comprise, for example, pharmaceutically acceptable additives, excipients, carriers, and the like, that may improve, for example, manufacturability, administration, taste, ingestion, uptake, and so on.

A therapeutically-effective amount can be administered to a patient in one or more doses sufficient to palliate, ameliorate, stabilize, reverse or slow the progression of the disease, or otherwise reduce the pathological consequences of the disease, or reduce the symptoms of the disease. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

Table A provides examples of routes of administration, dosage ranges and dosing schedules for the glycocalyx degraders and several of the compounds used in the methods of the invention. Other compounds used in the methods of the invention may be used in similar dosage ranges. The dosages for the compounds in Table A are calculated in relationship to body surface area, which for a typical 75 kg person is about 1.75 m².

TABLE A
Dosages and Administration Routes

| | Route of | | |
|---|---|---|---|
| Compound | Administration | Dosage range | Dosing Schedule |
| Glycocalyx | Intraserosal | 1-1000 IU**/ml | Prior to or together |
| degraders | | | with therapy/ |
| | | | treatment |
| LBH589 | Intraserosal, | 1-100 mg/m² | Continuous or |
| | intravenous | | intermittent |
| | or oral | | |
| AUY922 | Intraserosal, | 10-400 mg/m² | Continuous or |
| | intravenous | | intermittent |
| | or oral | | |
| LAQ824 | Intraserosal, | 1-100 mg/m² | Continuous or |
| | intravenous | | intermittent |
| | or oral | | |

**1 IU (international unit) is defined as the amount of the glycocalyx degrader that catalyzes the degradation of 1 micromole of glycocalyx per minute.

Yet a further aspect of the invention relates to a method to inhibit cancer stem cell self-renewal or formation by administering an inhibitor of glycocalyx formation or an agent that degrades glycocalyx for a time and in an amount to a patient to inhibit glycocalyx formation or degrade the glycocalyx of CSC in the patient and to thereby inhibit self-renewal or formation of said CSC, or to cause differentiation of the CSC and make them susceptible to killing, to prevent the catenae from undergoing spheroid formation, or any combination thereof, wherein the patient is also administered an effective amount of a compound selected from the group consisting LBH-589 (Panobinostat), NVP-AUY922, LAQ824 (NVP-LAQ824, Dacinostat), colchicine, brefeldin A, diphenyleneiodonium chloride or a combination thereof. This method can be administered and combined with other methods in the same manner as for other methods of treatment described herein.

B. Other Treatment Methods

Other treatment methods of the invention include a method to treat a serosal cancer which comprises (a) administering an anticancer regimen of the invention to a serosal cancer patient; (b) reviewing the results from one or more of the methods in section 5 above performed periodically with samples from said patient, and (c) altering the treatment regimen as needed and consistent with the information provided from those methods, i.e., by monitoring the serosal cancer stem cells present in a patient, a medical practitioner can make informed and personalized decisions about which therapeutic regimens would apply to that particular patient.

### 9. Potential Therapeutics

In addition to the gene signature information for catena, gene expression analysis gave significant information on the molecular pathways active in catena cells. Based on this information, Table B provides a list pathways active in catena and compounds that target those pathways as potentially effective therapeutics for serosal CSCs, and more particularly for ovarian CSCs. Underlined compounds have been tested for efficacy against catenae.

TABLE B
Catena Pathway Targeting Compounds

| Pathway | Compounds |
|---|---|
| Rho-ROCK | Y27632 |
| pathway | |
| DNA replication | 5-FU, ARA-C, mitomycin-C |
| c-met pathway | PF-02341066 |
| iNOS pathway | LNMMA |
| ROS pathway | L-buthionine Sulfoximine |
| ABC Transporters | Verapamil, Ningalin, Dexverapamil, SDZ PSC 833, SDZ 280-446, |
| | XR9051 |
| | GF120918, Nifedipine, Trifluoperazine, Midostaurin, Thapsigargin |
| | Zaprinast, MK-0457. |
| Metabolic inhibitors | Lovastatin acid, SB-201076, SB-204990, dichloroacetic acid/DCA, |
| | 2-deoxy-D-glucose (2DG), 3-bromopyruvate, 3-BrOP, 5-thioglucose, |
| AKT | Deguelin, GSK690693, MK-2206, Perfosine, Archexin, Triciribine, |
| | OSU-03012, INCB028060, PHT-472, AZD6244. |
| Cell cycle protein | PD-0332991, Olomoucine, Seliciclib., CEP-3891, CHIR-124, XL844, |
| inhibitors | |
| | PF-477736, UCN-01, LY2603618, AZD7762, CBT501, SCH 900776, |
| | Kinetin riboside |
| Receptor TK | Dasatinib, Sunitinib, Erlotinib/Tarceva, Nimotuzumab, |
| inhibitors | |
| | Cetuximab/Erbitux, Panitumumab, Trastuzumab, |
| | ZalutumumAb, PF-299804, AEE788, Vandetanib, JNJ-26483327, |
| | CI-1033, Lapatinib, PD-158780, BMS-599626 |
| | BMS-690514, PD153035, BIBW2992, ARRY-334543, AG1478 |
| | CL-387785, HKI-272, EKB-TKI, AZD8931. |
| | U0126, Sorafenib, PD0325901, INCB028060, |
| | TK1258, Maiatinib, Danusertib, SU6668, Regorafenib, PHA-665752, |
| | FP1039, AS703569, PD173074. |
| | 19D2, AMG-479, AVE-1642, BIIB022, Figitumumab, Di-diabody, |
| | H7C10, H710, MK-0646, R1507, BioG, IMC-A12, m610, BMS- |
| | 536,924, BMS-554417, EXEL-228, insm-18, NVP-ADW742, NVP- |
| | AEW541, OSI-906, Picropodophylin, PQ401, TAE226, BMS-754807, |
| | SU11274, A-923573, IGF1R antisense, IGF1R interference, m610, |
| | AZD6244, GSK1904529AXL-228, A-923573, INCB028060, 17- |
| | AAG, PU-H71 |
| | BMS-554417, OSI-906, BMS-754807, GSK1904529A, Capecitabine |
| | Etaracizumab, MEDI-522, Volociximab, Natalizumub, Cilengitide, |
| | S247, |
| | Cediranib, CHIR-258, Masitinib, Motesanib Diphosphate, Pazopanib |
| | Hydrochloride, Tandutinib, Vatalanib, Sunitinib Malate, Kit Mab, |
| | Axitinib, Imatinib Mesylate, Midostaurin, WBZ_4, Nilotinib, IMC- |
| | 41A10 |
| FSCN1/Fascin | Migrastatin, 2,3-dihydromigrastatin, Migrastatin core, Migrastatin ether |
| HAS2, Hyaluronan | 4-methyl-umbelliferone, 6,7-dihydroxy-4-methyl coumarin |
| | Hyaluronidase, rHuPH2, PEGPH20, Zaprinast, Brefeldin A, Mannose, |
| | 4-methylesculetin,5,7-dihydroxy-4-methyl coumarin. |
| HDAC | SAHA, Belinostat, JNJ-26481585, LAQ824, Panobinostat, |
| | Mocetinostat, |
| | Entinostat, PCI-24781, Trichostatin A, Vorinostat, SB939, Valproic |
| | Acid. |
| Hedgehog pathway | Cyclopamine, BMS-833923, GDC-0449, IPI-926, LDE225. |
| Heat shock protein | 17-AAG/Tanespimycin, Geldanamycin, 17-DMAG/Alvespimycin, |
| inhibitors | |
| | CNF-1010, IPI-504, IPI-493, KW-2478, KF25706, |
| | Cycloproparadicicol, |
| | Radicicol, Pochonin, PU24FC1, PU-DZ8, PU-H71, CNF-2024, SNX- |
| | 5422 |
| | STA-9090, VER-00063579, VER-49009, VER-50589, VER-52296 |
| | G3129, G3130, NMS-E973, PF-04929113, SNX-210, PU-H71, KU175 |
| | Celastrol, ATI3387, MPC-3100, AUY922. |
| | MAL3-101, VER-155008, Quercetin, KNK437 |
| MEK Targeting | 17-AAG, AMG 102, TAK-701, SCH-900105, XL-184, JNJ-38877605, |
| | GSK1363089, PF-04217903, PF-2341066, PHA-665752, SGX-523, |
| | SU11274, Compound 1, INCB028060, Foretinib, INCB028060, |
| | h224G11, MGCD265, PU-H71, NK4, MK-2461 |
| mTOR Targeting | Rapamycin, KU-55933, PI-103, Temsirolimus, BEZ235, Deforolimus, |
| | Everolimus, U0126, 852A, Imiquimod, XL765, Palomid 529, |
| | AZD8055, XL765, NVP-BEZ235, BGT226, GDC-0980, SB2312, PKI- |
| | 402 |
| NF-kB Targeting | Parthenolide, PDTC, Disulfiram, Olmesartan, Dithiocarbamate |
| Notch/Gamma | DAPT, RO4929097, (Z-LL)2-ketone, L-852646, MRK-003, GSI-I, |
| secretase Targeting | GSI-IX, GSI-XII, GSI-18, GSI-34, LY-411, 574, JC-34, JC-22, JC-22, |
| | MK-0752, IL-X, NLT1, NTL2, OMP-21M18, Dibenzazepine, z-Leu- |
| | leu-Nle-CHO, Notch3 siRNA, Begacestat |
| PDGFR targets | Dasatinib, JJ-101, Motesanib, Axitinib, Semaxanib, Sorafenib Tosylate, |
| | SU6668, Sunitinib, Masitinib, Pazopanib, Regorafenib, Linifanib, |
| | CHIR258, ABT-869, BIBF1120, CHIR-258, Imatinib, Mesylate, |
| | Tandutinib, Vatalanib, Leflunomide, Midostaurin, CP673, 451, IMG- |
| | 3G3, 2C5, 1 E10 |
| PI3K Targeting | LY294002, GDC-0941, GDC-0980, KU-55933, OSU-03012, PI-103, |
| | XL765, XL147, ZSTK474, AS041164, Deguelin, Halenaquinone, |
| | IC486068, PX-866, SF1126, WAY-266175, Wortmannin, BEZ235, |
| | XL765, NVP-BEZ235, BGT226, BKM120, CAL-120, SB2312, |
| | GSK2126458, PKI-402, Myoinositol, I3C, QLT0267 |
| Proteasome | Bortezomib/Velcade, NPI-0052, MG-132, Celastrol, CEP-18770, PF- |
| Inhibitors | 3084014, MLN9708, PR-047 |
| RAF (A-RAF, B- | 17-AAG, GDC-0879, Sorafenib Tosylate, PLX4032, XL281, RAF264, |
| RAF, C-RAF) | PU-H71 |
| Targeting | |
| SRC targeting | Dasatinib, PHA-665752, Saracatinib, Bosutinib, XL-228, AS703569 |
| Topoisomerase | Doxorubicin, Etoposide, 9-AC, Irinotecan, Camptothecan, 10- |
| (TOP1, TOP2) | Hydroxycamptothecin, 9-methoxycamptothecin, AR-67, Topotecan, |
| Targeting | NK012, Amsacrine, Teniposide, ICRF-193, Thaspine, Artemisini |
| Tubulin-alpha, beta | Epothilone B, dEpoB, 9,10-dehydro dEpob, Fludelone, Iso-oxazol |
| Targeting | fludelone, Paclitaxel, ABT-751, AVE8062, CA4P, DMXAA, |
| | EPC2407, MN-029, TZT-1027, ZD6126, BMS-247550, Patupilone, |
| | KOS-862, BMS-310705, ZK-EPO, KOS-1584, KOS-1584, Docetaxel, |
| | Taxotere |
| VEGFR, VEGF | Sunitinib, Avastin, IMC-18F1, IMC-1121B, PHA-665752, Axitinib, |
| Targeting | Midostaurin, Semaxanib, Sorafenib Tosylate, SU6668, SU6668, |
| | Pazopanib, BIBF1120, CHIR-258, Motesanib Diphosphate, Sorafenib |
| | Tosylate, Vatalanib, E-3810, AG13736, PTC299, Regorafenib, JJ-101, |
| | Brivanib, Linifanib, MGCD265, XL-184, Cediranib, Elesclomol, |
| | Enzastaurin, Vandetanib, XL-184, Vadimezan, GSK1363089, BMS- |
| | 690514, BMS-844203, Tivozanib, Midostaurin, RAF264, MGCD265, |
| | Aflibercept, CEP-3891, MK-2461 |

### 10. Monitoring and Staging Serosal Cancer

Yet further embodiments are drawn to methods of monitoring and/or staging serosal cancer in a subject. These methods comprise (a) preparing catenae from ascites obtained from a cancer patient; (b) detecting whether the catenae have one or more HAS2 mutations and/or express one or more HAS2 splice variants; and (c) correlating those mutations and/or variants with the presence and/or progression of cancer in a said patient. Further, one can identify or monitor for the presence of serosal cancer stem cells in a patient sample by (a) obtaining a cellular sample from a patient; (b) optionally, depleting that sample of leukocytes; (c) preparing DNA, RNA or both from the remainder of the sample; and (d) detecting whether the DNA, RNA or both has a HAS2 mutation or expresses a HAS2 splice variant, with the identification of a mutation or a splice variant indicating the presence of serosal cancer stem cells in the sample. By quantitating the amounts of such DNA or RNA, one can correlate the findings with the presence of serosal cancer and/or progression of a serosal cancer in the patient as well as efficacy of treatment.

These correlations include the ability to make an original diagnosis for the presence of serosal cancer, early detection of the cancer and its disease stage, the presence of cancer stem cells, the catenae content of a tumor, the aggressiveness of a tumor, the metastatic potential of a tumor and, the risk of metastasis of a tumor. Likewise, the HAS2 status of a patient can be used to stratify patients for hyaluronidase combination therapy and to correlate disease-free survival and response to therapy. A HAS2-based PCR assay can be integrated in clinical trials to follow the effect of chemotherapy on cancer stem cells and determine at early stages of the trial if the therapy is effective or not.

Samples for such assays can be ascites, preferable, but peripheral blood can be used as well. DNA or RNA can be directly amplified form ascites or blood samples and used in PCR method. Specific FISH (fluorescent in situ hybridization) probes for WT and variant mRNA can be used on blood smears or ascites samples spun on a diagnostic slide. The presence of these probes in the same cells can also be determined.

The HAS2 splice variant appears to be expressed in more of the ascites samples than solid tumors. Clinically, having ascites is poor prognosis so there is a correlation between variant expression and clinical outcome.

It will be appreciated by those skilled in the art that various omissions, additions and modifications may be made to the invention described above without departing from the scope of the invention, and all such modifications and changes are intended to fall within the scope of the invention, as defined by the appended claims. All references patents, patent applications or other documents cited are herein incorporated by reference in their entirety.

### EXAMPLES

Examples 1-12 of WO2011/057034 describe the development of an in vivo orthotopic ovarian cancer model, how inflammatory responses stimulate ovarian tumor growth, the isolation of tumor cells from NSG ascites, identification of catenae, the in vitro expansion of catenae, spheroid formation, and extensive characterization of the catenae and spheroids, including the ability to self-renew and to differentiate in vivo. The source and preparation of the various cell lines used below are described in detail in WO2011/057034.

The data indicate that catenae are clonally derived and do not develop by aggregation of diverse cell types. Catenae are uniform in morphology and in differentiation state, i.e., clonally pure CSCs can be obtained. While chain migration and a mesenchymal to catena transition are linked to tumor invasiveness, catenae provide a mechanism for rapid, symmetric CSC expansion. CSC expansion does not occur as efficiently in spheroids, and since spheroids contain proportionately fewer CSCs than catenae, it suggests that spheroids may structurally serve to protect CSCs and allow those CSCs to enter quiescence.

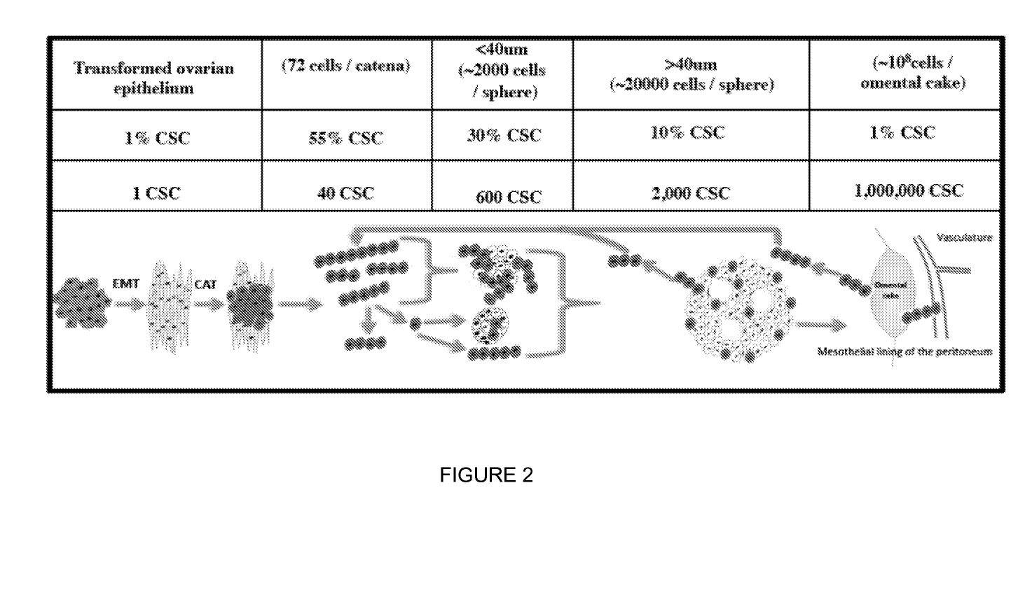

FIG. 2 provides a model of the catena-spheroid concept and the role of CSCs in the development of ovarian cancer. The initial transformation of ovarian (or fallopian) epithelium (green) progresses via an epithelial-mesenchymal and mesenchymal-catena transition. The catena cells (red) lose all attachment to extracellular matrix or peritoneal mesothelium but remain attached to each other following each round of symmetric division. At this point, the catena is composed predominantly of CSCs. The catena can release single cells that generate secondary catenae or form spheroids. The catenae can also rollup and form spheres which contain a >10 fold higher frequency of CSC than tumors growing as 2D monolayers or solid tumors. Spheroids can release new catenae or can attach to the mesothelial wall of the peritoneum to form omental cakes. Catenae may be released from solid tumors by a mesenchymal-catena transition and may reenter the peritoneal ascites or penetrate into blood vessels leading to more distant metastasis.

As further described in the examples of WO2011/057034, studies were conducted to determine the effects of basement membrane matrix on catena morphology (Example 20), and catena morphology was extensively analyzed by scanning and transmission electron microcopy (Example 21). The gene expression profiles of catenae were determined and are reported (Example 22), and the expression of upregulated catena genes was examined in other tissues (Example 23). The gene expression profiles of 366 advanced stage ovarian cancer patients and 10 normal ovary samples available through The Cancer Genome Atlas (TCGA; http://tcga.cancer.gov) were analyzed for expression of the upregulated and downregulated catena genes (Example 24). Catenae, spheroids and various cell lines were examined for differential miRNA expression (Example 25), for RTK phosphorylation (Example 26) and for surface phenotype (Example 27).

Moreover, the ability to culture catenae under defined conditions without serum (Example 28) has several advantages, including allowing identification of autocrine pathways, identification of secreted proteins, and isolation and characterization of exosomes, without contamination. For example, the secreted and exosomal proteins from catena were reported in Example 29. Finally, a HAS2 splice variant was reported (Example 30) as well as the expression of HAS2 and PDGFRA in ovarian cancer cell lines (Example 31).

### Example 1

### Catena and Spheroid Formation from Cancer Patient Ascites

### 1. Catena Formation

Serosal cancer samples from pleural, pericardial or ascites fluids containing tumor cells were obtained from cancer patients with metastatic cancer. Tumor cells were harvested by centrifugation at 1200 rpm for 10 min. The serosal fluid was removed and stored at −20° C. The harvested tumor cells were put into tissue culture flasks with serosal fluid from the same patient mixed 1:1 with serum-containing media. Free-floating chains of tumor cells were immediately observable under the microscope. The chains remained in suspension for many weeks. The tumor cells were cultured at 37° C. for several weeks and each week, the free-floating chains of cells in suspension were separated from the attached cells and replated into a new flask with the same combination of serosal fluid and serum-containing media. In these studies, as few as 100 of these free-floating cells from primary serosal tumor samples were able to form tumors in NSG mice in 3 months when injected subcutaneously. When injected intraperitoneally, these cells formed peritoneal tumors in NSG mice in 3-6 months with up to 10 ml of ascites containing free-floating tumor chains, liver metastasis and with solid tumors attached to peritoneal wall. Subsequent in vitro cultures of ascites samples from xenografts identified non-attached free-floating cells.

2. Generation of Spheroids from Catenae in Primary Serosal Tumor Samples:

To produce spheroids, catenae from primary serosal tumor samples growing in suspension were resuspended in serum-containing media mixed 50:1 with Matrigel and cultured at 37° C. The catenae from these primary serosal tumor samples rolled up to form organized tumor spheroids at about 5 days. Cultures were supplemented with serum containing media every week and after 2 weeks, tumor spheroids were observed to extrude catena into culture. Tumor spheroids can be maintained for weeks in vitro with this cell culture method.

### Example 2

### Screening Catenae for Drug Sensitivity

### 1. Methods

Ovcar3-GTL-derived catenae were tested for their ability to self-propagate in flat bottom 384-well microtiter plates (Corning). Cultures of Ovcar3-GTL catenae were mechanically or enzymatically dissociated to single cells. For mechanical dissociation, catena cultures were pipetted vigorously, an equal volume of M5-FCS media was added to decrease the viscosity, and the cells were pelleted. For enzymatic dissociation, catena cultures were incubated at 5 mg/ml collagenase IV (Invitrogen) for 10 min at 37° C. followed by centrifugation to pellet the cells. Cells were resuspended in M5-FCS to produce homogenous cultures of single cells which were seeded in 50 μL aliquots per well at the indicated cell densities and grown for the indicated times before addition of test compounds or other reagents.

To assess cell growth, cells were observed under the microscope and manually counted using a hemocytometer or were treated with alamarBlue® by adding 1/10 volume of alamarBlue reagent directly to the culture medium, incubating the cultures for a further 48 hours at 37° C. and measuring the fluorescence or absorbance. Both spectroscopic methods gave comparable results. The amount of fluorescence or absorbance is proportional to the number of living cells and corresponds to the cells metabolic activity. Fluorescence measurement is more sensitive than absorbance measurement and is measured by a plate reader using a fluorescence excitation wavelength of 540-570 nm (peak excitation is 570 nm) and reading emission at 580-610 nm (peak emission is 585 nm). Absorbance of alamarBlue® is monitored at 570 nm, using 600 nm as a reference wavelength. Larger fluorescence emission intensity (or absorbance) values correlate to an increase in total metabolic activity from cells.

Because the components of the pericellular glycocalyx were significantly removed prior to cell seeding by mechanical or enzymatic dissociation of catenae, the optimal time for adding compounds to ensure that the catenae had an established glycocalyx was determined and was found to be 3-6 days after seeding. For these experiments, 25 Ovcar3-GTL catena or 250 Ovcar3-GTL catena cells were seeded per well as described above. Test compounds were added at concentrations ranging from 12 pM to 100 μM (across the plate) on days one through six after seeding. Five days after adding the test compound, alamarBlue® was added to the cultures and culture absorbance was measured 48 hours later. No significant difference was observed between 25 or 250 cells in terms of drug sensitivity.

### 2. Proliferation Results—Selected Test Compounds

The results are shown in Table C for 23 test compounds on OvCar3-GTL catenae. This table sets out the identity of the test compound, the measured IC₅₀ in μM for samples in which the test compound was added one day after seeding (cells predominantly lacking a glycocalyx) and for samples in which the test compound was added six days after seeding (cells having an established or substantial glycocalyx). The final column of the table provides the increased fold of drug resistance from day 1 to day 6.

The results show that catena became resistant to 21 out of 23 agents in 6 days. Only bortezomib (Velcade®) and deguelin showed no differential sensitivity. The formation of glycocalyx in 6 days, for example, conferred more than 8,000,000-fold resistance in catenae to paclitaxel, fludelone and 9-10dEpoB. These results show that adding the compounds 1 day after cell seeding may lead to overestimation of the toxicity of compounds.

Another 6 compounds were tested which did not show any effect on catena cells, even at high concentrations. The compounds, 4-methylumbelliferone (4-MU), Y27632, 9-aminocamptothecin (9-AC), LNMMA, verapamil and dasatinib exhibited an IC₅₀ of 100 μM whether added on day one or day six post-seeding.

The foregoing total of 29 compounds were tested in parallel on ovarian cancer monolayer cells by seeding 100 Ovcar3 monolayer (epithelial) or 25 Ovcar5 monolayer (mesenchymal) cells in 384-well plates. Drugs were added 4 days after cell seeding and cell viability was analyzed by alamarBlue staining. In general, catena cells with an established glycocalyx were on average 4-8 fold more resistant to these compounds when compared to monolayers. However, this resistance was more pronounced for some compounds, including paclitaxel, iso-oxazole-fludelone, fludelone and 9-10dEpoB as shown in Table D. These four compounds were highly inhibitory to the Ovcar3 and Ovcar5 monolayer cells, having IC₅₀ values ranging from subnanomolar to no more than 50 nM, whereas catena cells (IC₅₀ 100 μM) were at least 2000-fold more resistant to these compounds.

The effect of these 29 compounds were also tested on established tumor spheroids. For these assays, 100 spheroid forming cells were seeded in 384-well plates and cultured for 11 days to allow the formation of tumor spheroids before adding drugs. Five days after adding the compound the cells were stained with alamarBlue and scored as above. Overall, spheroids showed the same pattern of drug resistance as catenae with an established glycocalyx. In the case of deguelin, spheroid formation conferred an additional 15-fold resistance to the cells, i.e., catena had an IC₅₀ of 0.025 μM whereas the spheroid IC₅₀ was 0.4 μM.

TABLE C
Ovcar3-GTL Catena Drug Sensitivity

| | | IC₅₀ (μM) | | |
|---|---|---|---|---|
| | | Addition | Addition | Increase in |
| | | on | on | Resistance |
| | Test Compound | Day 1 | Day 6 | Day6/Day 1 |
| 1 | paclitaxel | 0.000012 | 100 | 8,333,333 |
| 2 | fludelone | 0.000012 | 100 | 8,333,333 |
| 3 | 9,10 dehydroEpoB | 0.000012 | 100 | 8,333,333 |
| 4 | dEpoB | 0.000400 | 100 | 250,000 |
| 5 | iso-oxazole-fludelone | 0.003000 | 100 | 33,333 |
| 6 | Epo-B | 0.025000 | 100 | 4,000 |
| 7 | topotecan | 0.02 | 100 | 5,000 |
| 8 | Ara-C | 0.05 | 100 | 2,000 |
| 9 | daunarubacin | 0.006 | 0.8 | 133 |
| 10 | etoposide | 0.4 | 50 | 125 |
| 11 | PD-0332991 | 0.6 | 50 | 83 |
| 12 | mitomycin-C | 0.05 | 3 | 60 |
| 13 | 17AAG | 0.012 | 0.4 | 33 |
| 14 | 5-FU | 3 | 100 | 33 |
| 15 | doxorubicin | 0.025 | 0.8 | 32 |
| 16 | PF-02341066 | 0.8 | 25 | 31 |
| 17 | SAHA | 1.5 | 12 | 8 |
| 18 | parthenolide | 3 | 25 | 8 |
| 19 | LY294002 | 25 | 100 | 4 |
| 20 | lovastatin acid | 25 | 50 | 2 |
| 21 | rapamycin | 12 | 25 | 2 |
| 22 | deguelin | 0.025 | 0.025 | 1 |
| 23 | bortezomib | 0.013 | 0.013 | 1 |

TABLE D
Drug Sensitivity For Monolayers v. Catenae

| | IC₅₀ (μM) | | | Increased |
|---|---|---|---|---|
| | Ovcar5 | Ovcar3 | Ovcar3 | Resistance |
| Test Compounds | monolayer | monolayer | catena | of catena |
| Paclitaxel | 0.0250 | 0.0250 | 100 | 4,000 |
| Iso-oxazole-fludelone | 0.0120 | 0.0500 | 100 | 2,000 |
| Fludelone | 0.0004 | 0.0008 | 100 | 125,000 |
| 9,10 dehydroEpoB | 0.0004 | 0.0004 | 100 | 250,000 |

### 3. Proliferation Results—Pathway-Specific Inhibitors

Further screening assays were conducted as generally described in the methods section of this example using 48 pathway-specific inhibitors for pathways known or suspected as active in catena cells. The assays were conducted on catena cells with or without a glycocalyx coat and the IC₅₀ values were determined.

Table E provides the results for the 23 inhibitors that were inactive (generally much less toxic) against catena cells lacking a glycocalyx. For these compounds the IC₅₀ was greater than 10 μM. As expected, the presence of a glycocalyx coat did not appreciably change the IC₅₀ value.

Table F shows the IC₅₀ values and the fold of increased resistance to inhibitor for catena cells with glycocalyx coat for the remaining 25 compounds. These compounds were toxic against catena cells lacking a glycocalyx at concentrations <5 μM. The cellular toxicity of only one compound, PHA-665752, was unaffected by the presence of the glycocalyxcoat (fold resistance=1). The coat-associated resistance was >250-fold for four compounds, namely VX-680 (Aurora kinase inhibitor), XL880 (c-Met inhibitor) and BEZ235 (PI3K inhibitor) and BI2536 (PLK inhibitor).

Three compounds were identified as highly potent ovarian CSC inhibitors for having IC₅₀ values against catena cells with an established glycocalyx coat that were less than 0.2 μM. These compounds are two HDAC inhibitors (LAQ824 and LBH589) and an HSP90 inhibitor (NVP-AUY922).

TABLE E
Inactive Pathway Specific Inhibitors for Catena

| | | IC₅₀ (μM) | IC₅₀ (μM) |
|---|---|---|---|
| | Pathway Specific Inhibitor | No coat | With coat |
| 1 | CI-1040 (PD184352) | >25.0 | >25.0 |
| 2 | Gefitinib (Iressa) | >25.0 | >25.0 |
| 3 | Lenalidomide | >25.0 | >25.0 |
| 4 | Motesanib Diphosphate (AMG-706) | >25.0 | >25.0 |
| 5 | Pazopanib Hydrochloride | >25.0 | >25.0 |
| 6 | Y-27632 | >25.0 | >25.0 |
| 7 | SB 216763 | >25.0 | >25.0 |
| 8 | GDC-0449 (Vismodegib) | >25.0 | >25.0 |
| 9 | GSK1904529A | >25.0 | >25.0 |
| 10 | PF-04217903 | >25.0 | >25.0 |
| 11 | Vatalanib | >25.0 | >25.0 |
| 12 | GDC-0879 | >25.0 | >25.0 |
| 13 | ABT-888 (Veliparib) | 13.4 | >25.0 |
| 14 | Axitinib | 13.4 | >25.0 |
| 15 | U0126 | 13.4 | >25.0 |
| 16 | AV-951 (Tivozanib) | 13.4 | >25.0 |
| 17 | BIBF1120 (Vargatef) | 13.4 | 13.4 |
| 18 | Sorafenib Tosylate (Bay 43-9006) | 13.4 | 13.4 |
| 19 | Tandutinib (MLN518) | 13.4 | 13.4 |
| 20 | Masitinib (AB1010) | 13.4 | 13.4 |
| 21 | KU-55933 | 13.4 | 13.4 |
| 22 | PD0332991 | 13.4 | 13.4 |
| 23 | BAY 73-4506 (Regorafenib) | 13.4 | 13.4 |

TABLE F
Active Pathway Specific Inhibitors for Catena

| | | IC₅₀ (μM) | IC₅₀ (μM) | Fold |
|---|---|---|---|---|
| | Pathway Specific Inhibitor | No coat | With coat | resistance |
| 1 | LBH-589 (Panobinostat) | 0.01 | 0.05 | 4 |
| 2 | NVP-AUY922 | 0.00 | 0.05 | 24 |
| 3 | LAQ824 (NVP-LAQ824, | 0.01 | 0.20 | 16 |
| | Dacinostat) | | | |
| 4 | PHA-665752 | 3.14 | 3.14 | 1 |
| 5 | Vorinostat (SAHA) | 0.78 | 3.14 | 4 |
| 6 | BIBW2992 (Tovok) | 3.14 | 13.42 | 4 |
| 7 | CHIR-258 (Dovitinib) | 3.14 | 13.42 | 4 |
| 8 | Perifosine | 3.14 | 13.42 | 4 |
| 9 | Sunitinib Malate (Sutent) | 3.14 | 13.42 | 4 |
| 10 | MK-2206 | 3.14 | 13.42 | 4 |
| 11 | OSU-03012 | 3.14 | 13.42 | 4 |
| 12 | PI-103 | 0.78 | 13.42 | 16 |
| 13 | PF-2341066 (Crizotinib) | 0.78 | 13.42 | 16 |
| 14 | AP24534 (Ponatinib) | 0.78 | 13.42 | 16 |
| 15 | GDC-0941 | 3.14 | >25.0 | >8 |
| 16 | ZSTK474 | 3.14 | >25.0 | >8 |
| 17 | SU11274 (PKI-SU11274) | 3.14 | >25.0 | >8 |
| 18 | SGX-523 | 3.14 | >25.0 | >8 |
| 19 | JNJ-38877605 | 3.14 | >25.0 | >8 |
| 20 | MGCD-265 | 3.14 | >25.0 | >8 |
| 21 | PD0325901 | 0.20 | >25.0 | >125 |
| 22 | XL880 (GSK1363089EXEL- | 0.05 | 13.42 | 256 |
| | 2880) | | | |
| 23 | BEZ235 (NVP-BEZ235) | 0.05 | >25.0 | >500 |
| 24 | VX-680 (Tozasertib) | 0.05 | >25.0 | >500 |
| 25 | BI 2536 | <0.001 | 13.42 | >13000 |

### 4. Proliferation Results—LOPAC 1280

A library of 1280 compounds with well-documented pharmacological activities (LOPAC 1280) was subjected to HTS as described in the methods section of this example. Each compound was tested against three cellular entities (catena cells with or without the coat and established tumor spheroids) at 5 μM, 0.2 μM and 0.008 μM of compounds (with replicates). The most sensitive group among the three cellular entities was the catena cells without a glycocalyx coat: 67 hits were identified at 5 μM, twenty of which were still potent inhibitors at 0.2 μM and two of which were toxic at 0.008 μM. For spheroids, twenty compounds were toxic at 5 μM, 9 compounds at 0.2 μM and 1 compound at 0.008 μM.

Catena cells with a glycocalyx coat were the most drug resistant cellular entity: 12 compounds were found to be toxic at 5 μM. Only 3 of these compounds (colchicine, brefeldin A and diphenyleneiodonium chloride) could penetrate the glycocalyx and exert toxicity at 0.2 μuM. At 0.008 μM, no compounds were found that were toxic to catena cells with a glycocalyx

### 5. Morphological Results

Observing the catena cells under the microscope showed the presence of live, large single cells, i.e., cells arrested at mitosis, in cultures treated with high concentrations of compounds (100 μM topotecan, 25 μM rapamycin, 50 μM lovastatin acid, 100 μM iso-oxazole-fludelone, 100 μM fludelone, 100 μM ara-C, 100 μM 9-10dEpoB, 100 μM paclitaxel). When these cells were harvested and cultured in the absence of drugs, they re-entered the cell cycle.

Catenae treated with rapamycin formed tight spheroids with demarcated edges. These spheroids continued to grow in the presence of high concentrations of rapamycin (>50 uM) and retained their spheroid morphology. The formation of tight spheroids was also observed when catena cells were treated with SAHA (an HDAC inhibitor).

Catenae treated with 5-fluorouracil (5-FU) exhibited a morphological change resulting in formation of fused chains, suggesting that 5-FU may interfere with the tight and adherence junctions of catena. Similar structures were observed in ovarian cancer ascites and metastatic breast cancer patient samples. The change in the cell-to-cell junctions might also be a resistance mechanism where cells activate signaling pathways by increasing cell-to-cell attachment or more tightly control transport of molecules between cells.

Catena cells lost their polarity and formed free floating irregular cell aggregates when treated with high concentrations of verapamil. Similar morphological changes were observed when catena cells were treated with PEGylated or non-PEGylated bovine testis hyaluronidase at day 5 post seeding and cultured until day 10. When the coat is removed/destroyed by hyaluronidase catena cells lose their polarity and form irregular aggregates in vitro.

### Example 3

### Glycocalyx Analysis

The catena and spheroid cultures became increasingly viscous at high cell density. Without passage, the catena cultures became so viscous that harvesting the suspension cells was difficult even after a long incubation with collagenase-IV and/or strenuous mechanical dissociation, suggesting that the presence of a glycocalyx coat around the catenae and spheroids was generating the viscous (or mucinous) media. The cells and culture media were examined for the presence of mucins and hyaluronan.

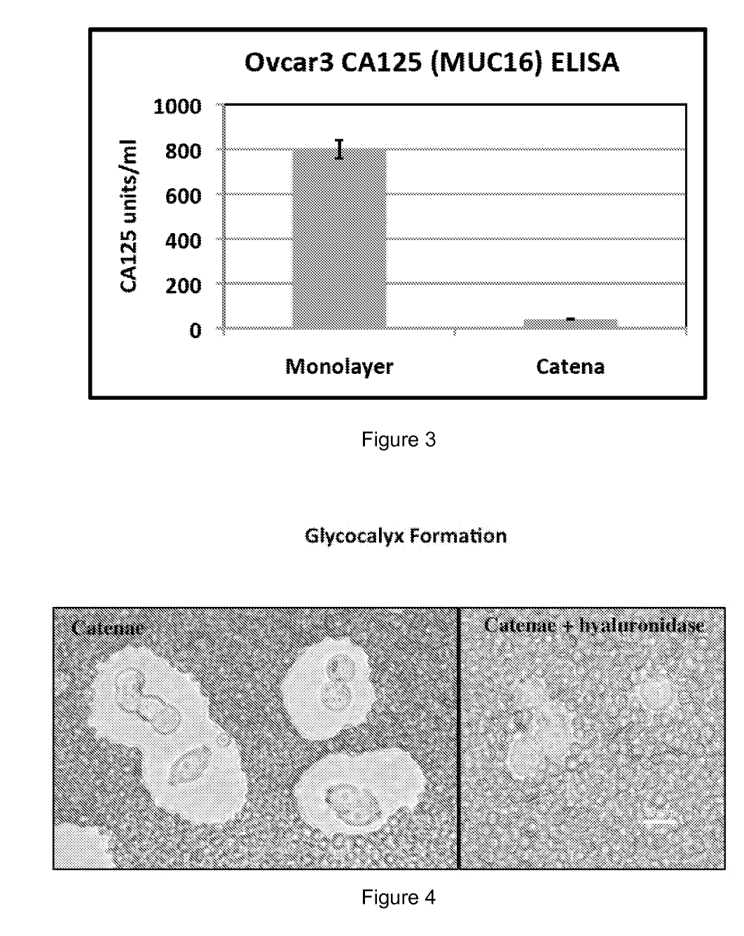

Initial FACS analysis for the mucin CA125 (the protein product of the MUC16 gene), a biomarker for different types of cancer, indicated that CA125 was not expressed on the surface of catenae. Likewise, ELISA experiments showed that CA125 was not secreted by catenae (FIG. 3). In contrast, Ovcar3-GTL epithelial cells were 98% positive for CA125 by FACS and secreted 800 units/ml of CA125 into culture media. For the ELISA, cell supernatants were collected by spinning the cultures at 300×g for 5 min to remove cells and assayed by CA125 ELISA using an automated instrument, ADVIA Centaur XP Immunoassay System (Siemens Healthcare Diagnostics Inc.).

Hyaluronan is a glycosaminoglycan found in extracellular matrix and functions to provide microenvironmental cues in a number of biological processes, including tumor development [Toole, 2004]. Supernatants prepared as above were treated with a few drops 10 mg/mL hyaluronidase (Sigma) in deionized water. The treatment rapidly reduced the viscosity of the supernatant, indicating hyaluronan was a major component of the viscous media.

To visualize the glycocalyx surrounding a catena, a particle exclusion experiment was conducted using red blood cells (RBCs). Catenae were mechanically dissociated by pipetting or by brief incubation with hyaluronidase as before. RBCs from human peripheral blood were added and the mixture was incubated overnight in culture media. The cells were observed under the light microscope for the presence of a glycocalyx separating catena cells from the RBCs. Mechanically-dissociated catenae mixed with RBCs had a glycocalyx coat extending up to 25 μm from the cell surface (FIG. 4, left panel), preventing direct catena-RBC cellular contact, whereas hyaluronidase-treated catena completely lacked a glycocalyx, allowing RBC-catena interaction (FIG. 4, right panel).

Because glycocalyx formation correlated with mesenchymal to amoeboid transition, the maintenance of glycocalyx integrity may be necessary for symmetric expansion of ovarian CSCs as catenae (and other serosal CSCs). For example, the glycocalyx may prevent integrin interactions with extracellular matrix, suggesting that removal of the glycocalyx should expose cell surface proteins and allow interactions with extracellular matrix or other attachment surfaces.

To investigate the how catena cells grow upon disruption of the glycocalyx, catenae were dissociated to single cells with hyaluronidase treatment and plated in tissue culture treated flasks with or without 10% hyaluronidase enzyme solution (10 mg/ml) to prevent the formation of glycocalyx. In parallel, catenae were dissociated mechanically and plated in the absence of hyaluronidase.

Mechanically-dissociated catenae remained in suspension where they proliferated rapidly to form free-floating chains of cell. Catenae dissociated to single cells with a brief treatment of hyaluronidase and plated in the absence of hyaluronidase enzyme no longer formed free floating chains but rather proliferated as irregular aggregates in suspension. In contrast, continuously hyaluronidase-treated cells attached to tissue culture plates and formed epithelial and mesenchymal monolayers. The results suggest that without a protective coat, ovarian CSCs are able to interact with attachment surfaces and respond to downstream differentiation stimuli.

The presence of different types of monolayers cells in these cultures validated the multilineage differentiation potential of ovarian CSCs from catenae. Epithelial monolayers were less frequently observed than mesenchymal cells indicating that more differentiation signals are needed to generate epithelial cancer cells than for mesenchymal cancer cells.

The glycocalyx of the catena has two major components, i.e., hyaluronan and collagen, which interact and form a stable complex. Western blot analysis showed a low molecular weight complex of collagen and hyaluronan (less than 20 kDa), detectable by anti-COL1A2 antibody. Briefly, the supernatant fraction of catena cell cultures was separated from the cells by centrifugation. The supernatant was run in an SDS-PAGE gel and blotted with the anti-COL1A2 antibody. This complex was sensitive to hyaluronidase treatment but was not affected by collagenase type 1, 2 or 4 treatment. This hyaluronan-collagen complex could be important for the formation of catena glycocalyx and drug resistance or metastatic potential conferred to catena cells by the glycocalyx.

The extracellular matrix of catena is isolated and analyzed for proteins present in catena glycocalyx as validated by deep sequencing and mass spectrometry of the secretome of catena cells.

Two important components of the extracellular matrix, elastin and fibronectin are not expressed by catenae. Laminin and collagen are major component of the catena glycocalyx along with hyaluronan. Hyaluronan and proteoglycans are linked and stabilized by HAPLN1 (hyaluronan proteoglycan link protein 1), HABP1 (hyaluronan binding protein 1) and HABP4 (hyaluronan binding protein 1) proteins. Each component of the glycocalyx is essential for the integrity of the coat and any changes in the composition effects the cell morphology and associated characteristics. When catena cells roll-up and form tumor spheroids, LUM (lumican), DCN (decorin) and JAM2 (junctional adhesion molecule 2), COL6A1 (collagen, type VI, alpha 1), COL6A2 (collagen, type VI, alpha 2), SGCG (sarcoglycan, gamma) genes are upregulated but HAPLN1, VCAN (versican) and GPC3 (glypican 3) genes are downregulated. Therefore, the glycocalyx of the spheroids are different than catena glycocalyx.

Table G lists extracellular matrix proteins that are upregulated and present in catenae (left column) and proteins that are downregulated in catenae (right column). The catena secretome fraction was analyzed for the presence or absence of these gene products and none of the down regulated genes were detected in that fraction.

TABLE G
Extracellular Matrix Proteins In Catenae

| Protein in | Downregulated |
|---|---|
| Glycocalyx | Gene in Catena |
| VCAN | ELN |
| NID1 | FN1 |
| NID2 | ACAN |
| MGP | DCN |
| LAMA5 | LUM |
| LAMB2 | TNXB |
| LAMC1 | AGRN |
| COL1A1 | |
| COL1A2 | |
| COL3A1 | |
| COL4A5 | |
| COL4A3BP | |
| COL5A2 | |
| COL6A3 | |
| COL6A1 | |
| HABP1/C1QBP | |
| HABP4 | |
| HAPLN1 | |

### Example 4

### Clonogenicity of Hyaluronidase-Treated Catenae

Catena cells were dissociated with hyaluronidase, allowed to attach to tissue culture plates and grown in the presence hyaluronidase for 7 days. Under these conditions, cells remained attached to tissue culture plates. The cells were harvested and subjected to an in vitro clonogenicity assay in the presence and absence of hyaluronidase. In parallel, mechanically-dissociated catena were subjected to the in vitro clonogenicity assay in the presence and absence of hyaluronidase.

Attached cells proliferated significantly slower than free-floating catenae and formed predominantly attached colonies with only a few cells “piling up” on mesenchymal and epithelial monolayers. The colony size was further reduced if hyaluronidase enzyme was included in the clonogenic assay. These results show that glycocalyx composed of hyaluronan is involved in maintaining the free-floating chain morphology and cancer stem cell characteristics of catenae.

### Example 5

### Combination Drug Screening

The glycocalyx around the catenae confers resistance to some therapeutic agents such as paclitaxel, fludelone and 9,10-dEpoB but not to others such as deguelin and bortezomib (See, Example 2). Since hyaluronan and collagen are major components of the catena glycocalyx, catena cells were tested to determine whether treatment with hyaluronidase and/or collagenase altered the drug resistance of catena cells.

### 1. PEGylation

Hyaluronidase and collagenase have short half lives in vivo and modification of these enzymes by attachment of polyethylene glycol (PEG; the process being PEGylation) has been shown to increase the stability of enzymes from minutes to several hours. To PEGylate these enzymes, alpha-methoxy-omega-carboxylic acid succinimidyl ester polyethylend glycol (PEG MW 20,000) (MeO-PEG-NHS) was used by mixing 100 mg MeO-PEG-NHS with 0.5 mL 10 mg/mL bovine testis hyaluronidase (25000 U/mL) and 15 ml PBS. The mixture was incubated at 4° for 48 hrs on a rotator. For PEGylation of collagenase, 0.5 mL of 10 mg/mL collagenase 1 (2500 U/mL) was substituted for the hyaluronidase.

The PEGylated and non-PEGylated samples, reduced and non-reduced, were run by protein gel electrophoresis and stained with Coomassie blue. The expected increases in band size were observed, including the addition of multiple PEG moieties.

To examine whether PEGylation inhibited enzymatic activity, catenae were treated with PEGylated or non-PEGylated hyaluronidase [as described above]. Both treatments caused aggregation of catena cells. Addition of collagenase 1 to catena cultures does not affect the morphology of those cells, and similarly, addition of PEGylated collagenase 1 did not have any effect on catena morphology.

### 2. Drug Screening

Twenty-five catena cells were seeded into 384-well plates. After 5 days, cells were treated with either PEGylated hyaluronidase, PEGylated collagenase or both for 10 minutes at 37° C. Without removing the enzymes, paclitaxel was added over a series of dilutions, followed by alamarBlue addition on day 9 with absorbance measured two days later. The IC₅₀ for paclitaxel alone was unchanged in the presence of PEGylated collagenase. Treatment of the cultures with PEGylated hyaluronidase prior to adding paclitaxel decreased the IC₅₀ by 2.5 fold and treating with the combination PEGylated enzymes, decreased the IC₅₀ by 16 fold for paclitaxel, a value comparable to that obtained when paclitaxel was added to plates 1 day after cell seeding, i.e., when the catena cells lacked any substantial amount of glycocalyx.

### REFERENCES

Bapat S A, Mali A M, Koppikar C B, Kurrey N K. Stem and progenitorlike cells contribute to the aggressive behavior of human epithelial ovarian cancer. Cancer Res. 2005 Apr. 15; 65(8):3025-9.
Becker L., Prewett T. L., Spaulding G. F. and Goodwin T. J. Threedimensional growth and differentiation of ovarian tumor cell line in high aspect rotating-wall vessel: morphologic and embryological considerations. J. Cell. Biochem. 51 (1993), pp. 283-289.
Davidson B. Biological characteristics of cancers involving the serosal cavities. Crit Rev Oncog. 2007; 13:189-227.
Di Maria G U, Comba P, Malignant Pleural Mesothelioma: The Puzzling Role of Gene-Environment Interaction. Chest. 2004; 125:1604-1607.
Jemal A, Siegel R, Ward E, Hao Y, Xu J, Murray T, Thun M J. Cancer statistics, 2008. C A Cancer J Clin. 2008 March-April; 58(2):71-96.
Ludwig, T. E., Bergendahl, V., Levenstein, M. E., Yu, J., Probasco, M.D. & Thompson. J. A. 2006. Feeder-independent Culruer of Human Embryonic Stem Cells. Nature Med 3: 637-646.
Markman M, Markman J, Webster K, Zanotti K, Kulp B, Peterson G, Belinson J. Duration of response to second-line, platinum-based chemotherapy for ovarian cancer: implications for patient management and clinical trial design. J Clin Oncol. 2003 Sep. 1; 21(17):3194-200. Epub 2003 Jul. 14.
Olive P. L., Durand R. E. Drug and radiation resistance in spheroids: cell contact and kinetics. Cancer Metastasis Rev., 1994: 13: 121-138.
Ozols R F, Bundy B N, Greer B E, Fowler J M, Clarke-Pearson D, Burger R A, Mannel R S, DeGeest K, Hartenbach E M, Baergen R; Gynecologic Oncology Group. Phase III trial of carboplatin and paclitaxel compared with cisplatin and paclitaxel in patients with optimally resected stage III ovarian cancer: a Gynecologic Oncology Group study.
Park S M, Gaur A B, Lengyel E, Peter M E. The miR-200 family determines the epithelial phenotype of cancer cells by targeting the E-cadherin repressors ZEB1 and ZEB2. Genes Dev. 2008 Apr. 1; 22(7):894-907.
Szotek P P, Pieretti-Vanmarcke R, Masiakos P T, Dinulescu D M, Connolly D, Foster R, Dombkowski D, Preffer F, Maclaughlin D T, Donahoe P K. Ovarian cancer side population defines cells with stem cell-like characteristics and Mullerian Inhibiting Substance responsiveness. Proc Natl Acad Sci USA. 2006 Jul. 25; 103(30):11154-9.
Toole B P. Hyaluronan: from extracellular glue to pericellular cue. Nat Rev Cancer. 2004 Jul.; 4(7):528-39.
Zhang S, Balch C, Chan M W, Lai H C, Matei D, Schilder J M, Yan P S, Huang T H, Nephew K P. Identification and characterization of ovarian cancer-initiating cells from primary human tumors. Cancer Res. 2008 Jun. 1; 68(11):4311-20.

1-10. (canceled)

11. A method to treat serosal cancer in a patient undergoing chemotherapy which comprises administering a hyaluronan synthase inhibitor, a hyaluronidase, a collagenase, or a combination thereof, for a time and in an amount to augment said chemotherapy, or to improve or increase patient survival time, or to cause remission of symptoms,
wherein said chemotherapy comprises administering an effective amount of a compound selected from the group consisting LBH-589 (Panobinostat), NVP-AUY922, LAQ824 (NVP-LAQ824, Dacinostat), colchicine, brefeldin A and diphenyleneiodonium chloride, or a combination thereof, to the patient.

12. The method of claim 11, wherein any one of said hyaluronan synthase inhibitor, hyaluronidase or collagenase is PEGylated or otherwise modified to increase its half life in vivo.

13. The method of claim 11, wherein said administering a hyaluronan synthase inhibitor, a hyaluronidase, a collagenase, or a combination thereof, is done before, concurrently with, overlapping or after said chemotherapy chemotherapy.

14. The method of claim 11, wherein serosal cancer is ovarian cancer or metastatic ovarian cancer.

15. The method of claim 11, which further comprises treating said cancer by administering one or more radiation treatments to said patient

16. The method of claim 11 which further comprises periodically monitoring samples from said patient to assess whether catenae or spheroids can be obtained therefrom, and altering the treatment regimen as needed consistent with the presence or absence of catenae or spheroids in said samples.

17. A method for treating serosal cancer in a patient which comprises administering a therapeutically-effective amount of a compound selected from the group consisting LBH-589 (Panobinostat), NVP-AUY922, LAQ824 (NVP-LAQ824, Dacinostat), colchicine, brefeldin A and diphenyleneiodonium chloride, or a combination thereof, to the patient.

18. The method of claim 17, wherein serosal cancer is ovarian cancer or metastatic ovarian cancer.

19. The method of claim 18, wherein said compound is LBH-589 (Panobinostat), LAQ824 (NVP-LAQ824, Dacinostat) or brefeldin A.

20. The method of claim 17, which further comprises treating said cancer by administering one or more radiation treatments to said patient.

21. The method of claim 17 which further comprises periodically monitoring samples from said patient to assess whether catenae or spheroids can be obtained therefrom, and altering the treatment regimen as needed consistent with the presence or absence of catenae or spheroids in said samples.

22. A method to inhibit cancer stem cell (CSC) self-renewal or formation in a patient which comprises administering an inhibitor of glycocalyx formation or an agent that degrades glycocalyx for a time and in an amount to said patient to inhibit glycocalyx formation or degrade the glycocalyx of CSC in the patient and to thereby inhibit self-renewal or formation of said CSC, or to cause differentiation of the CSC and make them susceptible to killing, to prevent catenae from undergoing spheroid formation, or any combination thereof,
wherein said patient is also administered an effective amount of a compound selected from the group consisting LBH-589 (Panobinostat), NVP-AUY922, LAQ824 (NVP-LAQ824, Dacinostat), colchicine, brefeldin A and diphenyleneiodonium chloride, or any combination thereof.

23. The method of claim 22, wherein said inhibitor or agent is PEGylated or otherwise modified to increase its half life in vivo.

24. The method of claim 22, wherein serosal cancer is ovarian cancer or metastatic ovarian cancer.

25. The method of claim 22, which further comprises administering one or more radiation treatments to said patient.

26. The method of claim 22 which further comprises periodically monitoring samples from said patient to assess whether catenae or spheroids can be obtained therefrom, and altering the treatment regimen as needed consistent with the presence or absence of catenae or spheroids in said samples.
